SEQUENCE LISTING (57 sequences)

SEQ ID NO: 1 - protein, 301 aa, Homo sapiens
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVPLFPPPTPLEPSPTLPDLDVVADTGGEEDTEDQGLTGDEAEPFLDQSGGPGAPTTPRKLPSRPPPHYPGIKRKRSAPTSRKLLSDKPQDFQIRVQVIEGRQLPGVNIKPVVKVTAAGQTKRTRIHKGNSPLFNETLFFNLFDSPGELFDEPIFITVVDSRSLRTDALLGEF

SEQ ID NO: 2 - protein, 517 aa, Homo sapiens
PRQFHQLAAQGPQECLVRIYIVRAFGLQPKDPNGKCDPYIKISIGKKSVSDQDNYIPCTLEPVFGKMFELTCTLPLEKDLKITLYDYDLLSKDEKIGETVVDLENRLLSKFGARCGLPQTYCVSGPNQWRDQLRPSQLLHLFCQQHRVKAPVYRTDRVMFQDKEYSIEEIEAGRIPNPHLGPVEERLALHVLQQQGLVPEHVESRPLYSPLQPDIEQGKLQMWVDLFPKALGRPGPPFNITPRRARRFFLRCIIWNTRDVILDDLSLTGEKMSDIYVKGWMIGFEEHKQKTDVHYRSLGGEGNFNWRFIFPFDYLPAEQVCTIAKKDAFWRLDKTESKIPARVVFQIWDNDKFSFDDFLGSLQLDLNRMPKPAKTAKKCSLDQLDDAFHPEWFVSLFEQKTVKGWWPCVAEEGEKKILAGKLEMTLEIVAESEHEERPAGQGRDEPNMNPKLEDPRRPDTSFLWFTSPYKTMKFILWRRFRWAIILFIILFILLLFLAIFIYAFPNYAAMKLVKPFS

SEQ ID NO: 3 - protein, 127 aa, Homo sapiens
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLP

SEQ ID NO: 4 - protein, 21 aa, Homo sapiens
LADGLSSLAPTNTASPPSSPH

SEQ ID NO: 5 - protein, 12 aa, Homo sapiens
LADGLSSLAPTN

SEQ ID NO: 6 - protein, 8 aa, Artificial
  Amino acid sequence from spectrin, subject to calpain cleavage
EEVYGMMK

SEQ ID NO: 7 - protein, 809 aa, Artificial
  Midi-dysferline comprises dysferlin Nt domain, polypeptide encoded by exon 40a and dysferlin Ct domain
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVPLFPPPTPLEPSPTLPDLDVVADDVSLLSPGEDVLIDIDDKEPLIPIQLADGLSSLAPTNTASPPSSPHEEEFIDWWSKFFASIGEREKCGSYLEKDFDTLKVYDTQLENVEAFEGLSDFCNTFKLYRGKTQEETEDPSVIGEFKGLFKIYPLPEDPAIPMPPRQFHQLAAQGPQECLVRIYIVRAFGLQPKDPNGKCDPYIKISIGKKSVSDQDNYIPCTLEPVFGKMFELTCTLPLEKDLKITLYDYDLLSKDEKIGETVVDLENRLLSKFGARCGLPQTYCVSGPNQWRDQLRPSQLLHLFCQQHRVKAPVYRTDRVMFQDKEYSIEEIEAGRIPNPHLGPVEERLALHVLQQQGLVPEHVESRPLYSPLQPDIEQGKLQMWVDLFPKALGRPGPPFNITPRRARRFFLRCIIWNTRDVILDDLSLTGEKMSDIYVKGWMIGFEEHKQKTDVHYRSLGGEGNFNWRFIFPFDYLPAEQVCTIAKKDAFWRLDKTESKIPARVVFQIWDNDKFSFDDFLGSLQLDLNRMPKPAKTAKKCSLDQLDDAFHPEWFVSLFEQKTVKGWWPCVAEEGEKKILAGKLEMTLEIVAESEHEERPAGQGRDEPNMNPKLEDPRRPDTSFLWFTSPYKTMKFILWRRFRWAIILFIILFILLLFLAIFIYAFPNYAAMKLVKPFS

SEQ ID NO: 8 - protein, 21 aa, Artificial
  Mutated polypeptide  S1485A and P1487A
LADGLSSLAPTNTAAPASSPH

SEQ ID NO: 9 - protein, 21 aa, Artificial
  Mutated polypeptide S1485A ; P1487A ; S1489G
LADGLSSLAPTNTAAPASGPH

SEQ ID NO: 10 - protein, 21 aa, Artificial
  Mutated polypeptide S1476Y
LADGLYSLAPTNTASPPSSPH

SEQ ID NO: 11 - protein, 21 aa, Artificial
  Mutated polypeptide S1476A ; S1477G
LADGLAGLAPTNTASPPSSPH

SEQ ID NO: 12 - protein, 21 aa, Artificial
  Mutated polypeptide T1481A
LADGLSSLAPANTASPPSSPH

SEQ ID NO: 13 - protein, 21 aa, Artificial
  Mutated polypeptide T1483K
LADGLSSLAPTNKASPPSSPH

SEQ ID NO: 14 - protein, 21 aa, Artificial
  Mutated polypeptide S1488Y
LADGLSSLAPTNTASPPTSPH

SEQ ID NO: 15 - protein, 17 aa, Artificial
  Mutated polypeptide Del PTNT
LADGLSSLAASPPSSPH

SEQ ID NO: 16 - protein, 20 aa, Artificial
  Mutant Exon 40a sequence Del P1480
LADGLSSLATNTASPPSSPH

SEQ ID NO: 17 - protein, 21 aa, Artificial
  Mutated polypeptide P1480S
LADGLSSLALTNTASPPSSPH

SEQ ID NO: 18 - protein, 21 aa, Artificial
  Mutated polypeptide A1472Y
LYDGLSSLAPTNTASPPSSPH

SEQ ID NO: 19 - protein, 18 aa, Artificial
  Mutated polypeptide Del LAD
GLSSLAPTNTASPPSSPH

SEQ ID NO: 20 - DNA, 63 bp, Homo sapiens
cttgcagacggtctgtcgagcttggcccccactaacacggcttctcctccatccagtcctcat

SEQ ID NO: 21 - DNA, 2430 bp, Artificial
  Nucleotide sequence of midi-dysferlin
atgctcagagtcttcattctgtatgccgagaacgtccacacccccgacacagacattagcgacgcctactgctccgcggtcttcgccggcgtgaaaaagaggacaaaagtgatcaagaatagcgtgaaccccgtctggaatgagggatttgagtgggatctgaagggcattcctctggaccaaggcagcgagctgcatgtggtggtcaaggaccacgagacaatgggaagaaataggtttctgggcgaagctaaggtgcctctgagagaggtgctggctaccccctctctgagcgcctccttcaacgctcctctgctcgacacaaagaagcagcctaccggcgcttctctggtgctgcaagtgagctacacccctctgcccggcgctgtgcctctcttcccccctcctacacctctggagccttcccctacactgcccgacctcgacgtcgtggccgatgacgtgtctctgctgagccccggcgaggatgtgctgatcgacattgacgacaaggaacctctgatccctatccaactggccgatggactgtcctccctcgcccccaccaatacagcttccccccctagcagcccccatgaggaggagttcatcgactggtggagcaaattcttcgcttccatcggcgagagagaaaagtgtggcagctatctggaaaaagacttcgataccctcaaggtgtatgacacccagctggagaatgtcgaggcttttgaaggactgagcgacttctgcaacacattcaagctctatagaggcaaaacccaagaggagaccgaagacccctccgtgatcggcgagtttaagggactgttcaagatttaccctctgcccgaggaccccgccatccctatgccccctaggcaattccatcaactggccgcccaaggcccccaagaatgtctggtgaggatttacatcgtcagagccttcggactgcagcccaaagatcccaacggcaagtgcgatccctatatcaagatcagcatcggcaaaaagtccgtcagcgaccaagacaactacatcccttgcacactggagcccgtgttcggcaagatgttcgagctgacatgtacactgcccctcgaaaaggacctcaagatcaccctctatgactacgatctgctctccaaagatgaaaagatcggcgagaccgtggtggatctcgaaaacagactgctgtccaagttcggagctagatgtggactgccccagacctactgcgtgtccggacccaaccagtggagggaccagctgagaccctcccaactgctgcatctgttttgtcagcagcatagagtcaaagctcccgtgtatagaaccgatagagtcatgttccaagacaaggaatattccattgaggagatcgaggctggaaggattcctaacccccacctcggacccgtggaggagaggctggctctgcacgtgctgcagcaacaaggcctcgtccccgagcacgtcgagtccagacccctctatagccccctccaacccgacatcgagcaaggcaaactccagatgtgggtggacctcttccccaaagctctcggcagacccggcccccccttcaatattacccctaggagagctagaaggttctttctgagatgtatcatctggaatacaagagacgtgattctggacgacctcagcctcaccggcgagaagatgtccgacatctacgtcaagggctggatgatcggcttcgaggagcacaaacagaagaccgatgtgcactatagatctctgggcggagaaggcaatttcaactggaggttcatcttccctttcgattatctccccgctgagcaagtgtgcacaatcgccaagaaggacgccttttggaggctggacaagaccgagagcaagatccccgccagagtggtgttccagatctgggataacgacaagttttccttcgatgactttctgggaagcctccagctggatctgaataggatgcctaagcccgccaagacagccaagaagtgcagcctcgatcaactcgatgacgccttccatcccgagtggttcgtgagcctcttcgagcagaaaaccgtgaaaggctggtggccttgtgtggccgaggaaggagagaagaagattctggccggaaagctggaaatgacactggagatcgtggctgaatccgagcatgaagagaggcccgccggccaaggaagagacgaacccaatatgaaccccaagctggaggaccctagaagacccgatacatcctttctgtggttcaccagcccctacaagaccatgaaatttatcctctggaggaggtttagatgggctatcatcctctttatcattctgttcattctgctgctcttcctcgccatctttatctacgcctttcccaactacgccgctatgaaactggtcaagcctttttcctga

SEQ ID NO: 22 - DNA, 47 bp, Artificial
  pcDNADysf11delN “Forward”
gcccctcatccccatccagaacacggcttctcctccatccagtcctc

SEQ ID NO: 23 - DNA, 50 bp, Artificial
  pcDNADysf11delN “Reverse”
gatggaggagaagccgtgttctggatggggatgaggggctccttgtcatc

SEQ ID NO: 24 - DNA, 46 bp, Artificial
  pcDNADysf11delC “Forward”
gtcgagcttggcccccactgaggaagagttcatcgattggtggagc

SEQ ID NO: 25 - DNA, 48 bp, Artificial
  Primer pcDNADysf11delC “Reverse”
ccaatcgatgaactcttcctcagtgggggccaagctcgacagaccgtc

SEQ ID NO: 26 - DNA, 45 bp, Artificial
  pcDNADysf11delN1 “Forward”
cctcatccccatccagagcttggcccccactaacacggcttctcc

SEQ ID NO: 27 - DNA, 48 bp, Artificial
  pcDNADysf11delN1 “Reverse”
gttagtgggggccaagctctggatggggatgaggggctccttgtcatc

SEQ ID NO: 28 - DNA, 45 bp, Artificial
  pcDNADysf11delN2 “Forward”
gcttgcagacggtctgtcgaacacggcttctcctccatccagtcc

SEQ ID NO: 29 - DNA, 45 bp, Artificial
  pcDNADysf11delN2 “Reverse”
ggaggagaagccgtgttcgacagaccgtctgcaagctggatgggg

SEQ ID NO: 30 - DNA, 835 bp, Artificial
  Nucleotide sequence of GFP used for labelling
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggccgctcgagtctagagggcccgcggttcgaacaaaaactcatctcagaagaggatctgaatatgcataccggtcatcatcaccatcaccattgagtttaaacccgctgatcagcct

SEQ ID NO: 31 - DNA, 711 bp, Artificial
  Nucleotide sequence for mCherry plasmid
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 32 - DNA, 9596 bp, Artificial
  Nucleotide sequence for Dysferlin 1
atgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctga

SEQ ID NO: 33 - DNA, 6306 bp, Artificial
  Nucleotide sequence for dysferlin 11
atgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccagcttgcagacggtctgtcgagcttggcccccactaacacggcttctcctccatccagtcctcatgaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctga

SEQ ID NO: 34 - DNA, 63 bp, Artificial
  Mutated Exon 40a sequence encoding polypeptide 1
cttgcagacggtctgtcgagcttggcccccactaacacggctgctcctgcatccagtcctcat

SEQ ID NO: 35 - DNA, 63 bp, Artificial
  Mutant Exon40a sequence encoding polypeptide 2
cttgcagacggtctgtcgagcttggcccccactaacacggctgctcctgcatccggtcctcat

SEQ ID NO: 36 - DNA, 63 bp, Artificial
  Mutant Exon40a sequence encoding polypeptide 3
cttgcagacggtctgtacagcttggcccccactaacacggcttctcctccatccagtcctcat

SEQ ID NO: 37 - DNA, 63 bp, Artificial
  Mutant Exon40a sequence encoding polypeptide 4
cttgcagacggtctggcgggcttggcccccactaacacggcttctcctccatccagtcctcat

SEQ ID NO: 38 - DNA, 63 bp, Artificial
  Mutant Exon40a sequence encoding polypeptide 5
cttgcagacggtctgtcgagcttggcccccgctaacacggcttctcctccatccagtcctcat

SEQ ID NO: 39 - DNA, 63 bp, Artificial
  Mutant Exon40a sequence encoding polypeptide 6
cttgcagacggtctgtcgagcttggcccccactaacaaggcttctcctccatccagtcctcat

SEQ ID NO: 40 - DNA, 63 bp, Artificial
  Mutant Exon40a sequence encoding polypeptide 7
cttgcagacggtctgtcgagcttggcccccactaacacggcttctcctccaaccagtcctcat

SEQ ID NO: 41 - DNA, 51 bp, Artificial
  Mutant Exon40a sequence encoding polypeptide 8
cttgcagacggtctgtcgagcttggccgcttctcctccatccagtcctcat

SEQ ID NO: 42 - DNA, 60 bp, Artificial
  Mutant Exon40a sequence encoding polypeptide 9
cttgcagacggtctgtcgagcttggccactaacacggcttctcctccatccagtcctcat

SEQ ID NO: 43 - DNA, 60 bp, Artificial; identical to SEQ ID NO: 42
  Mutant Exon40a sequence encoding polypeptide 10

SEQ ID NO: 44 - DNA, 63 bp, Artificial
  Mutant Exon40a sequence encoding polypeptide 11
ctttatgacggtctgtcgagcttggcccccactaacacggcttctcctccatccagtcctcat

SEQ ID NO: 45 - DNA, 54 bp, Artificial
  Mutant Exon40a sequence encoding polypeptide 12
ggtctgtcgagcttggcccccactaacacggcttctcctccatccagtcctcat

SEQ ID NO: 46 - DNA, 12 bp, Artificial
  Nucleotide sequence of pcDNA Dysf1
atccaggaggaa

SEQ ID NO: 47 - protein, 4 aa, Artificial
  pcDNA Dysf1 amino acids sequence
IQEE

SEQ ID NO: 48 - DNA, 75 bp, Artificial
  pcDNA Dysf 11 nucleotide sequence
atccagcttgcagacggtctgtcgagcttggcccccactaacacggcttctcctccatccagtcctcatgaggaa

SEQ ID NO: 49 - protein, 25 aa, Artificial
  pcDysf11 amino acid sequence
IQLADGLSSLAPTNTASPPSSPHEE

SEQ ID NO: 50 - DNA, 42 bp, Artificial
  pcDNADysf11 delN nucleotide sequence
atccagaacacggcttctcctccatccagtcctcatgaggaa

SEQ ID NO: 51 - protein, 14 aa, Artificial
  pcDNA Dysf11 delN amino acids sequence
IQNTASPPSSPHEE

SEQ ID NO: 52 - DNA, 42 bp, Artificial; identical to SEQ ID NO: 50
  pcDNA Dysf11 delC nucleotide sequence

SEQ ID NO: 53 - protein, 15 aa, Artificial
  pcDNA Dysf11 delC amino acids sequence
IQLADGLSSLAPTEE

SEQ ID NO: 54 - DNA, 57 bp, Artificial
  pcDNA Dysf11 delN1 nucleotide sequence
atccagagcttggcccccactaacacggcttctcctccatccagtcctcatgaggaa

SEQ ID NO: 55 - protein, 19 aa, Artificial
  pcDNA Dysf11 delN1 amino acid sequence
IQSLAPTNTASPPSSPHEE

SEQ ID NO: 56 - DNA, 60 bp, Artificial
  pcDNA Dysf11 delN2 nucleotide sequence
atccagcttgcagacggtctgtcgaacacggcttctcctccatccagtcctcatgaggaa

SEQ ID NO: 57 - protein, 20 aa, Artificial
  pcDNA Dysf11 delN2 amino acids sequence
IQLADGLSNTASPPSSPHEE

(19) United States
(12) Patent Application Publication
(10) Pub. No.: US 2024/0228564 A1
(43) Pub. Date: Jul. 11, 2024

(54) COMPOSITION FOR TREATING DYSFERLINOPATHY

(71) Applicants: UNIVERSITÉ D'AIX-MARSEILLE, Marseille Cedex 07 (FR); INSTITUT NATIONAL DE LA SANTÉ ET DE LA RECHERCHE MÉDICALE, Paris (FR)

(72) Inventors: Marc BARTOLI, Allauch (FR); Sébastien COURRIER, Simiane-Collongue (FR); Océane BALLOUHEY, Marseille (FR)

(73) Assignees: UNIVERSITÉ D'AIX-MARSEILLE, Marseille Cede 07 (FR); NSTITUT NATIONAL DE LA SANTÉ ET DE LA RECHERCHE MÉDICALE, Paris (FR)

(21) Appl. No.: 18/558,654

(22) PCT Filed: Apr. 28, 2022
(86) PCT No.: PCT/EP2022/061409

§ 371 Date: Nov. 2, 2023

(30) Foreign Application Priority Data
May 3, 2021 (EP) 21305569.2

(51) Int. Cl. C07K 14/47; A61K 48/00; A61P 21/00; C12N 15/86

(52) U.S. Cl. CPC C07K 14/4707; A61K 48/005; A61P 21/00; C12N 15/86; C12N 2750/14143

(57) ABSTRACT

Gene therapy for treating dysferlinopathy. More particularly, a polynucleotide sequence including several domains of dysferlin, or functional variants thereof, and a viral vector for gene therapy including at least a polynucleotide sequence, which includes exon 40a of the coding sequence of dysferlin. Also, a pharmaceutical composition including the viral vector.

### FIELD

The present invention pertains to gene therapy for treating dysferlinopathy. More particularly, the invention relates to a polynucleotide sequence comprising several domains of dysferlin, or functional variants thereof, it also relates to a viral vector for gene therapy comprising at least a polynucleotide sequence of the invention exon 40a of the coding sequence of dysferlin.

### BACKGROUND

Dysferlinopathies are autosomal recessive disorders caused by mutations in the dysferlin (DYSF) gene, encoding the dysferlin protein. Dysferlin is a modular type II transmembrane protein containing seven calcium sensor C2 domains that play a key role in muscle membrane repair. DYSF mutations lead to a wide range of muscular phenotypes, in particular Miyoshi myopathy (MM), that affects distal muscles and limb girdle muscular dystrophy type 2B (LGMD2B or LGMDR2) characterized by proximal weakness. Symptoms generally appear at the end of childhood and, although disease progression is typically slow, walking impairment eventually result.

Various therapeutic approaches for treating myopathies or dysferlinopathies have been proposed so far, although no curative treatment for dysferlinopathies exist.

WO 2020/123645 discloses a combination therapy for treating muscular dystrophy, wherein adenovirus gene therapy vectors comprise a polynucleotide encoding a first polypeptide, or a first RNA, and a second polypeptide, or a second RNA. This combination therapy involves both gene restoration and reduction of symptoms associated with a number of secondary cascades such as fibrosis.

WO 2018/170408 discloses an adeno-associated virus vector delivery of muscle specific micro-dystrophin, to treat muscular dystrophy. The gene therapy vectors are used to raise muscular strength and/or prevent fibrosis in patients affected by muscular dystrophy.

US 2020/0010521 discloses a truncated dysferlin nucleic acid and protein designed as “Nanodysferlin”.

WO 2011/054659 discloses an exon-skipping therapy for dysferlinopathies, by a process comprising a step of preventing the splicing of one or more exons coding for amino acid sequences responsible of dysferlin dysfunction. Said exon is in particular exon 32. Complementary antisense oligonucleotides used for correcting said splicing are also disclosed.

Barthelemy et al (“Translational research and therapeutic perspectives in dysferlinopathies”, Mol. Med. 17(9-10) 875-882, sept-oct, 2011) is a review describing therapeutic perspectives in dysferlinopathies. Barthelemy discloses a “mini-dysferlin” comprising the last two C2 domains of dysferlin and the transmembrane domain.

There is therefore a strong need for active therapy of dysferlinopathies.

The inventors have now shown that a protein comprising an amino acid sequence encoded by exon 40a of dysferlin protein is a key factor in the membrane repair process, and participates in other muscle cell functions, like muscle cell membrane protection and protein vesicle trafficking.

The present invention therefore relates to a nucleic acid sequence encoding for essential domains of dysferlin, including a nucleic acid sequence, such as exon 40a (GenBank EF015906), encoding for a cleavage site by calpain. A nucleic acid sequence of the invention is therefore useful to restore muscle cells repairing function in patients with dysferlinopathy.

Krahn et al (“Exclusion of mutations in the dysferlin alternative exons 1 of DYSF-v1, 5a and 40a in a cohort of 26 patients”, Gen. Test. Mol. Biomarkers, 2010, Feb. 14(1); 153-4, DOI 10.1089/gtmb.2009.0131) disclose a screening of mutations causing primary dysferlinopathies in three known alternative exons, DYSF-v1, 5a and 40a. The authors conclude that no disease-causing mutation was identified in said alternative exons, demonstrating a low frequency of disease-causing mutations in these exons.

Redpath et al (“Calpain cleavage within dysferlin exon 40a releases a synaptotagmin-like module for membrane repair”, MBoC, vol. 25, Oct. 1, 2014) disclose that injury-activated cleavage of dysferlin is mediated by the ubiquitous calpains via a cleavage motif encoded by alternately spliced exon 40a.

### SUMMARY

The inventors have now developed a nucleic acid sequence comprising, or consisting of, from its 5′ to 3′ extremity:
a) a nucleic acid sequence encoding for at least 100 amino acids of the dysferlin N-terminal amino acids No 1 to 301 (SEQ ID No 1) or for an amino acid sequence exhibiting at least 80% identity with SEQ ID No 1,
b) a nucleic acid sequence encoding for a polypeptide comprising a cleavage site by calpain, and
c) a nucleic acid sequence encoding for the dysferlin C2F, C2G and TM domains (SEQ ID No 2), for an amino acid sequence exhibiting at least 80% identity with said SEQ ID No 2 or for an amino acid sequence comprising at least 100 amino acids of SEQ ID No 2.

A nucleic acid sequence according to the invention codes for a polypeptide which is useful to prevent or abolish symptoms of dysferlinopathy, when injected to a patient in need thereof. A nucleic acid sequence according to the invention codes for a polypeptide useful to restore cell membrane repair, cell membrane protection and/or vesicle trafficking. In particular, a nucleic acid sequence according to the invention codes for a polypeptide able to restore cell membrane repair.

In a particular embodiment of the invention, a nucleic acid sequence of the invention encodes for a protein designated as “Midi-dysferlin” (SEQ ID No 4), a functional fragment thereof or a functional variant thereof.

The inventors have also developed a recombinant expression vector comprising at least a nucleic acid sequence according to the invention. A recombinant expression vector according to the invention can be used as a gene therapy vector. It is submitted that a recombinant expression vector according to the invention at least allows the restoration of the phenotype of a dysferlin deficient cell in an animal model, and may restore the “calpain cleavage activation” of dysferlin in a patient in need thereof.

The invention is based on the surprising discovery that the polypeptide encoded by exon 40a is strongly involved in the dysferlin repairing process of muscle cells membranes through the presence of a cleavage site by calpain, and on the observation that some mutations in said exon 40a sequence do not abolish the functional property of said peptide to form a cleavage site by calpain.

The inventors consequently defined a domain comprising a cleavage site by calpain, wherein said domain is either a polypeptide encoded by exon 40a, a functional fragment thereof or a functional variant thereof, or a polypeptide domain known in the art as susceptible to be cleaved by calpain.

The present invention therefore provides a gene replacement therapy aimed to at least partially restore dysferlin functions. A vector of the present invention therefore helps to reduce or prevent at least one symptom of a dysferlinopathy.

### BRIEF DESCRIPTION OF THE FIGURES

FIGS. 1A, 1B and 1C show the location difference between N-ter and C-ter part of dysferlin11 transcript. 1A represents the dysferlin, the GFP and Hamlet-1 binding site and the cleavage site by calpain. 1B and 1C represent respectively dysferlin labeling performed on transfected and uninjured (1B) or transfected and injured (1C). In both 1B and 1C, upper images represent murine myoblasts C2C12 transfected with dysferlin1 (which does not comprise Exon 40a) and lower images represent C2C12 cells transfected with dysferlin11 (which does comprise Exon 40a). In both 1B and 1C, left images represent GFP labeling, middle images represent Hamlet-1 labeling and right images represent merge labeling. DAPI was used as a nucleus marker (blue). All images were captured by an apotome microscope. Scale bar, 100 μM.

FIGS. 2A, 2B and 2C illustrate the importance of the N-terminal part of DYSF exon 40a for calpain cleavage. 2A represents the different dysferlin11 constructs created to identify cleavage site by calpain in DYSF exon 40a. Constructs were built by PCR fusion. 2B represents a western blot performed with proteins from HEK cells transfected with dysferlin constructs and injured with ionomycin and cells scrapers 24 h post-transfection. Hamlet was used to detect dysferlin and dysferlin cleaved (respectively upper and middle part of 2B). Actin was used for normalization (lower 2B). 2C represents the normalized ratio of cleaved dysferlin for each construction, results show that only dysferlin transcript 11 was able to be cleaved by calpain. For 2B and 2C, “a” is for Dysf1, “b” for Dysf11, “c” for Dysf11deIN, “d” for Dysf11delC, “e” for Dysf11delN1, “f” for Dysf11deIN2.

FIGS. 3A, 3B and 3C, of example 1, illustrate the importance of dysferlin11 transcript for muscle cell membrane repair. 3A: Membrane repair assay performed on wild-type myoblasts (WT, C25), dysferlin-null myoblasts (DYSF-null, AB320) and transfected dysferlin-null myoblasts (DYSF-null+mCherry-dysferlin1, DYSF-null+mCherry-dysferlin11, DYSF-null+mCherry-dysferlin1+mCherry-dysferlin11) in the presence of Ca²⁺ and membrane-impermeable dye FM 1-43. Fluorescence images were taken every second for 5 minutes. The damage was induced at t=10 sec. Scale bar, 10 μM. White arrows indicate injury site. 3B: Summary data for membrane repair assay. The black arrow indicates injury time point. Data are mean±SEM (Standard error of the mean) for n=20 cells. Statistical analysis was done with Mann-Whitney test (NS: non-significant, *: p-value<0.05, **: p-value<0.01). 3C presents the results of statistical analysis.

FIGS. 4A and 4B, of example 1, shows that dysferlin11 transcript participates in the protection of membrane myoblasts from mechanical stress induced by osmotic shock and represents the percentage of cell survival as a function of time (4A) and the results of statistical analysis (4B). Osmotic shock assay performed on wild-type myoblasts (WT, C25), dysferlin-null myoblasts (DYSF-null, AB320) and transfected dysferlin-null myoblasts (DYSF-null+GFP-dysferlin1, DYSF-null+GFP-dysferlin11, DYSF-null+GFP-dysferlin1+GFP-dysferlin11) in hypotonic solution (ultrapure distilled water). Fluorescence images were taken every minute for 30 minutes. Medium change was done at t=1 minute. Data are mean±SEM (Standard error of the mean) for n=10. For each experiment, at least 20 cells were observed. Statistical analysis was done with Mann-Whitney test (NS: non-significant, *: p-value<0.05, **: p-value<0.01).

FIG. 5 illustrates the intensity of transferrin in each cell (wherein a dot represents a cell) in the different reaction conditions (WT, DYSF-null or transfected DYSF-null cells) and shows that dysferlin11 transcript participates in protein vesicle trafficking in muscle cell. The upper part represents “Ratio Trf 488 intensity” of transferrin assay performed, from left to right, on wild-type myoblasts (WT, C25), dysferlin-null myoblasts (DYSF-null, AB320) and transfected dysferlin-null myoblasts (DYSF-null+mCherry-dysferlin1, DYSF-null+mCherry-dysferlin11, DYSF-null+mCherry-dysferlin1+mCherry-dysferlin11). Myoblasts were incubated for 30 min with transferrin coupled with Alexa Fluor 488 (pulse, dark grey, left side of each of the five tested conditions) and for 30 min with transferrin-free medium (pulse-chase, light grey, right side of each of the five tested conditions). For each condition, transferrin intensity was measured in more than 180 cells observed in three experiments. Statistical analysis was done with Mann-Whitney test (NS: non-significant, *: p-value<0.05, **: p-value<0.01) and Cohen test. The lower part of FIG. 5 represents Cohen test for each condition.

FIGS. 6A and 6B represent respectively the schematic construction of “midi-dysferlin”, said polypeptide comprising, from its Nt to Ct extremities, a C2A domain, a polynucleotide encoded by Exon 40a, C2F and C2G domains and transmembrane domain TM (6A) and a western blot (6B) performed with proteins from wild-type myoblasts (WT, C25), dysferlin-null myoblasts (DYSF-null, AB320) and transfected dysferlin-null myoblasts (DYSF-null+Midi-dysferlin), Hamlet was used to detect dysferlin and midi-dysferlin, GAPDH was used for normalization.

FIGS. 7A and 7B, of example 2, represent respectively a membrane repair assay (7A) performed on wild-type myoblasts (WT, C25, upper images), dysferlin-null myoblasts (DYSF-null, AB320, middle images) and transfected dysferlin-null myoblasts (DYSF-null+mCherry-midi-dysferlin, lower images) in the presence of Ca²⁺ and membrane-impermeable dye FM 1-43. Fluorescence images were taken every second for 5 minutes, wherein images represent from left to right, images taken at 0, 60, 120, 180 and 360 seconds. The damage was induced at t=10 sec. Scale bar is 10 μM. White arrows indicate injury site. 7B represents summary data for membrane repair assay, wherein the ratio DF/F0 is represented as a function of time (in seconds), the upper black curve represents DYSF-null, the intermediate grey curve represents DYSF-null+midi-dysferlin, the lower black curve represents WT. Black arrow «lesion» indicate injury time point. Data are mean±SEM (Standard error of the mean) for n=20 cells. Statistical analysis was done with Mann-Whitney test (NS: non-significant, *: p-value<0.05, **: p-value<0.01).

FIG. 8, of example represents the cell survival ratio as a function of time in an osmotic shock assay performed on wild-type myoblasts (WT, C25), dysferlin-null myoblasts (DYSF-null, AB320) and transfected dysferlin-null myoblasts (DYSF-null+GFP-midi-dysferlin) in hypotonic solution (ultrapure distilled water). Fluorescence images were taken every minute for 30 minutes. Medium change was done at t=1 minute. Data are mean±SEM (Standard error of the mean) for n=10. For each experiment, at least 20 cells were observed. Statistical analysis was done with Mann-Whitney test (NS: non-significant, *: p-value<0.05, **: p-value<0.01).

FIG. 9 of example 2, represents transferrin intensity in a transferrin assay performed on wild-type myoblasts (WT, C25, left), dysferlin-null myoblasts (DYSF-null, AB320, middle) and transfected dysferlin-null myoblasts (DYSF-null+mCherry-midi-dysferlin, right). Myoblasts were incubated for 30 min with transferrin coupled with Alexa Fluor 488 («pulse” dots, left) and for 30 min with transferrin-free medium (“pulse-chase” right). For each condition, transferrin intensity was measured in more than 290 cells observed in three experiments. Statistical analysis was done with Mann-Whitney test (NS: non-significant, *: p-value<0.05, **: p-value<0.01). The lower part represents Cohen's coefficient for each condition with respectively: for WT: WT pulse-chase minus WT pulse, for dysferlin-null myoblasts: DYSF-null pulse-chase minus DYSF-null pulse, and for transfected dysferlin-null myoblasts: midi-dysferlin pulse-chase minus midi-dysferlin pulse.

FIGS. 10A, 10B and 10C, of example 3, are histograms showing the properties of DYSF p.Y1159X/p.Y1159X mice injected with AAV comprising a nucleotide sequence of midi-dysferlin according to the invention. 10A represents the muscle strength (in g) of mouse transfected with PBS (left box) or with an AAV vector encoding for midi dysferlin. 10B represents the percentage of fibers positive for dysferlin in the cells from (from left to right) left quadriceps, right quadriceps, left gastrocnemius and right gastrocnemius. 10C represents the percentage of centronucleated fibers in the cells from (from left to right) PBS-injected left quadriceps, right quadriceps, left gastrocnemius and right gastrocnemius; AAV-midi dysferlin injected left quadriceps, right quadriceps, left gastrocnemius and right gastrocnemius.

### DETAILED DESCRIPTION

In a first aspect, the present invention relates to an isolated nucleic acid sequence comprising, or consisting of, from its 5′ to 3′ extremity:
a) a nucleic acid sequence encoding the dysferlin amino acids No 1 to 301 (SEQ ID No 1), for an amino acid sequence exhibiting at least 80% identity with SEQ ID No 1, or for an amino acid sequence comprising at least 100 amino acids of SEQ ID No 1.
b) a nucleic acid sequence encoding for a polypeptide comprising a cleavage site by calpain, and
c) a nucleic acid sequence encoding for the dysferlin C2F, C2G and TM domains (SEQ ID No 2), for an amino acid sequence exhibiting at least 80% identity with said SEQ ID No 2 or for an amino acid sequence comprising at least 100 amino acids of SEQ ID No 2.

The gene encoding human dysferlin (DYSF, Online Mendelian Inheritance in Man (OMIM) gene number 603009, 2p13, GenBank NM_003494.2) encodes several transcripts, the most common transcript is composed of 55 exons and alternative splicing events generate transcript diversity, such as exon 1, exon 5a and exon 40a (GenBank EF015906).

From its N-terminal to its C-terminal extremity, the human dysferlin protein comprises:
dysferlin amino acids No 1 to 301 comprising C2A and C2B domains, wherein said C2A and C2B domains bind to phospholipids, with C2A domain binding to phospholipids in a calcium dependent manner,
dysferlin amino acids No 381 to 1992 comprising C2C, C2D, C2E, C2F and C2G domains,
dysferlin amino acids No 2045 to 2067 comprising a transmembrane domain (TM). At the C-terminal extremity of the protein, dysferlin amino-acids 2068 to 2080 are usually located in the extra-cellular compartment

By “a nucleic acid sequence encoding for at least 100 amino acids” of a given nucleotide or amino-acid sequence, it is intended a nucleic acid sequence encoding for at least 100, 150, 200, 250, 280, 290 or 300 amino acids of said amino acid sequence.

By “a nucleic acid sequence encoding for a fragment of at least 100 amino acids” of a given nucleotide or amino-acid sequence, it is intended a nucleic acid sequence encoding for at least a fragment of 100, 150, 200, 250, 280, 290 or 300 amino acids of said amino acid sequence.

By “exhibiting at least 80% identity”, it is meant that said sequences exhibit at least 80% identity after optimal overall alignment, that is to say by global alignment between two sequences giving the highest percentage identity. between them. The optimal global alignment of two sequences can in particular be carried out according to the Needleman-Wunsch algorithm, well known to those skilled in the art (Needleman & Wunsch, “A general method applicable to the search for similarities in the amino acid sequences of two proteins”, J. Mol. Biol., 48 (3): 443-53).

Proteins encoding by a nucleic acid according to the invention comprise, or consist of an amino acid sequence having at least 80%, advantageously at least 81%, 82%, 83%, 84%, 85%, 86%, 87%, 88%, 89%, 90%, 91%, 92%, 93%, 94%, 95%, 96%, 97%, 98% or 99% identity with the amino acid sequence SEQ ID No 1 after global alignment optimal. Advantageously, the proteins according to the invention comprise, or consist of an amino acid sequence exhibiting at least 99.1%, 99.2%, 99.3%, 99.4%, 99.5%, 99.6%, 99.7%, 99.8% or 99.9% identity with said SEQ ID No 1

In a particular embodiment of this aspect, a nucleic acid sequence according to the invention encodes for a polypeptide exhibiting, at least partially, functional properties of said polypeptide having an amino acid sequence SEQ ID No 1.

By “exhibiting, at least partially, functional properties of said polypeptide” it is intended a polypeptide exhibiting at least 10%, 15%, 20%, 30%, 40% or 50% of the functional properties of said polypeptide, expressed as a measure or an estimation of the percentage of the functional property of a polypeptide having an amino acid sequence SEQ ID No 1.

By “functional properties” of a polypeptide exhibiting at least 80% identity with SEQ ID No 1, it is intended:
an ability to bind to membrane surfaces phospholipids, in particular to phosphoinositide and to phosphatidylserine, possibly in a calcium dependent manner, and
an ability to bind to proteins involved in membrane repair, in particular to tubulin and to MG53.

Calpains (code CAPN for Calcium Activated Neutral Proteases) are non-lysosomal cysteine proteases exhibiting a cytosolic papain-like activity controlled by calcium. Calpains are expressed ubiquitously in mammals. Today, 14 different calpains are described, including calpain 1 and calpain 2. The calpain proteolytic system includes the calpain proteases and the small regulatory subunit CAPNS1, also known as CAPN4.

By “a nucleic acid sequence encoding for a polypeptide comprising a cleavage site by calpain” it is intended a nucleic acid sequence encoding for a polypeptide which is cleaved by the calpain proteolytic enzyme, which is naturally present in the cells, under appropriate reaction conditions. Among calpains susceptible to cleave said polypeptide, calpain 1 and/or calpain 2 are preferred.

Appropriate reaction conditions are defined as follows:
an incubation of myoblasts (at 70% of confluency) with ionomycine (30 μM) at room temperature for 3 minutes and then
an incubation in PBS 1× supplemented with calcium 1 mM at 37° C. during 3 minutes to allow muscle repair mechanism.

Proteins of the treated myoblasts are then extracted and analysed by SDS-PAGE.

It has been shown that the cleavage of dysferlin by calpain leads to the presence of mini-dysferlin. Therefore, the presence of a cleavage site by calpain can be demonstrated by the presence of mini-dysferlin after said cleavage reaction. More generally, the presence of a cleavage site by calpain can be demonstrated by the presence of a protein of the appropriate size after said cleavage reaction.

It is estimated that an ability to being cleaved by calpain, even at a low level, is acceptable for a polypeptide encoded by a nucleic acid sequence of the invention. In particular, an ability to being cleaved by calpain of at least 5%, 10%, 15%, 20%, 30%, 40% or 50% of the property of the wild-type polypeptide encoded by exon 40a is acceptable.

Among nucleic acid sequences encoding for a polypeptide comprising a cleavage site by calpain, a particular embodiment of the invention relates to nucleic acid sequences encoding for the cleavage site by calpain of spectrin (SEQ ID No 6).

Among nucleic acid sequences encoding for a polypeptide comprising a cleavage site by calpain, a particular embodiment of the invention relates to nucleic acid sequences encoding for:
a polypeptide exhibiting the amino acid sequence (SEQ ID No 4) or an amino acid sequence exhibiting at least 80% identity with SEQ ID No 4,
a polypeptide exhibiting the amino acid sequence (SEQ ID No 5) or an amino acid sequence exhibiting at least 80% identity with SEQ ID No 5,
a polypeptide exhibiting the amino acid sequence (SEQ ID No 6) or an amino acid sequence exhibiting at least 80% identity with SEQ ID No 6.

More particularly, nucleic acid sequences encoding for a polypeptide exhibiting the amino acid sequence (SEQ ID No 4) or for an amino acid sequence exhibiting at least 80% identity with SEQ ID No 4 are preferred.

The inventors have shown that some polypeptides exhibiting mutated sequences of SEQ ID No 4 are still able to be cleaved by calpain, these polypeptides exhibit an amino acid sequence chosen among: SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18 and SEQ ID No 19.

Consequently, in a particular embodiment of the invention, a nucleic acid sequence encoding for a polypeptide comprising a cleavage site by calpain encodes for a polypeptide exhibiting an amino acid sequence chosen among: SEQ ID No 4, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18 and SEQ ID No 19.

In a preferred embodiment, a nucleic acid sequence encoding for a polypeptide comprising a cleavage site by calpain encodes for a polypeptide exhibiting an amino acid sequence SEQ ID No 4.

By “a nucleic acid sequence encoding for the dysferlin C2F, C2G and TM domains (SEQ ID No 2) it is intended a nucleic acid sequence encoding for the amino acids No 1563 to 2080 of dysferlin, which comprises domains C2F, C2G and TM of said dysferlin protein. SEQ ID No 2 includes amino acids 2068 to 2080 which are located in the extra-cellular compartment.

According to this aspect, a nucleic acid sequence encoding for at least 100 amino acids of the dysferlin N-terminal amino acids of SEQ ID No 2 or for an amino acid sequence exhibiting at least 80% identity with SEQ ID No 1, exhibits, at least partially, functional properties of SEQ ID No 2.

In a particular embodiment, the present invention relates to an isolated nucleic acid sequence comprising, or consisting of, from its 5′ to 3′ extremity:
a) a nucleic acid sequence encoding for at least 100 amino acids of the dysferlin N-terminal amino acids No 1 to 301 (SEQ ID No 1) or for an amino acid sequence exhibiting at least 80% identity with SEQ ID No 1, wherein said nucleic acid sequence encodes for the amino acid sequence of C2A domain (SEQ ID No 3), or for an amino acid sequence exhibiting at least 80% identity with SEQ ID No 3.
b) a nucleic acid sequence encoding for a polypeptide comprising a cleavage site by calpain, and
c) a nucleic acid sequence encoding for the dysferlin C2F, C2G and TM domains (SEQ ID No 2), for an amino acid sequence exhibiting at least 80% identity with said SEQ ID No 2 or for an amino acid sequence comprising at least 100 amino acids of SEQ ID No 2.

In another particular embodiment, the present invention relates to an isolated nucleic acid sequence comprising, or consisting of, from its 5′ to 3′ extremity:
i. a nucleic acid sequence encoding for at least 100 amino acids of the dysferlin N-terminal amino acids No 1 to 301 (SEQ ID No 1) or for an amino acid sequence exhibiting at least 80% identity with SEQ ID No 1,
ii. a nucleic acid sequence encoding for a polypeptide comprising a cleavage site by calpain, wherein said nucleic acid sequence encodes for a polypeptide comprising a cleavage site by calpain, said polypeptide comprising or consisting of the amino acid sequence SEQ ID No 4, a functional fragment thereof comprising at least a sequence SEQ ID No 5, or an amino acid sequence exhibiting at least 80% identity with SEQ ID No 6,
iii. a nucleic acid sequence encoding for the dysferlin C2F, C2G and TM domains (SEQ ID No 2), for an amino acid sequence exhibiting at least 80% identity with said SEQ ID No 2 or for an amino acid sequence comprising at least 100 amino acids of SEQ ID No 2.

In another particular embodiment, the present invention relates to an isolated nucleic acid sequence comprising, or consisting of, from its 5′ to 3′ extremity:
i. a nucleic acid sequence encoding for at least 100 amino acids of the dysferlin N-terminal amino acids No 1 to 301 (SEQ ID No 1) or for an amino acid sequence exhibiting at least 80% identity with SEQ ID No 1,
ii. a nucleic acid sequence encoding for a polypeptide comprising a cleavage site by calpain, wherein said nucleic acid sequence encoding for a cleavage site by calpain is nucleic acid sequence SEQ ID No 20, corresponding to dysferlin exon 40a, a nucleic acid sequence exhibiting at least 80% identity with SEQ ID No 20 or a nucleic acid sequence comprising at least 30 nucleotides of SEQ ID No 20,
iii. a nucleic acid sequence encoding for the dysferlin C2F, C2G and TM domains (SEQ ID No 2), for an amino acid sequence exhibiting at least 80% identity with said SEQ ID No 2 or for an amino acid sequence comprising at least 100 amino acids of SEQ ID No 2.

In a more particular embodiment, said nucleic acid sequence encoding for an amino acid sequence comprising at least 100 amino acids of SEQ ID No 2 is a nucleic acid sequence encoding for an amino acid sequence comprising at least a fragment of at least 200 amino acids of SEQ ID No 2, at least 300, 400 or at least 400 amino acids of SEQ ID No 2.

Said dysferlin C2F, C2G and TM domains (SEQ ID No 2), an amino acid sequence exhibiting at least 80% identity with said SEQ ID No 2 or an amino acid sequence comprising at least 100, at least 200, at least 300, 400 or at least 400 amino acids of SEQ ID No 2 are able to bind to cell membranes.

To show that said protein, or protein fragment, is addressed to the membrane of the tissues in which it is expressed, it is possible to show that it belongs to the protein extract from these tissues which are associated with membranes.

As an example, the following experimental protocol is performed: muscular tissue is homogenized, using a glass-Teflon Potter homogenizer, in ice-cold 50 mM Tris-HCl buffer, pH 7.5, containing 150 mM NaCl, 5 mM MgCl2, inhibitors of proteases and 1 mM EGTA. The homogenates are centrifuged at 100,000 g, at 4° C. for 1 h. The pellets solubilized in 50 mM Tris-HCl buffer containing 0.9% Lubrol-PX and 1 mM EGTA and centrifuged at 100,000 g for 30 min at 4° C. fractions are analyzed on SDS-polyacrylamide (7.5%) gels and transferred to nitrocellulose. The blots are stained with Ponceau red, and the presence of the protein of interest is revealed by using a specific antibody.

In another particular embodiment, the present invention relates to an isolated nucleic acid sequence comprising, or consisting of, from its 5′ to 3′ extremity:
i. a nucleic acid sequence encoding for the dysferlin C2A domain (SEQ ID No 3),
ii. a nucleic acid sequence corresponding to exon 40a (SEQ ID No 20), and
iii. a nucleic acid sequence encoding for the dysferlin C2F, C2G and TM domains (SEQ ID No 2).

In another particular embodiment, the present invention relates to an isolated nucleic acid sequence comprising, or consisting of the nucleic acid sequence SEQ ID No 21, or a nucleic acid sequence exhibiting at least 80%, 81%, 82%, 83%, 84%, at least 85%, 86%, 87%, 88%, 89%, 90%, 91%, 92%, 93%, 94%, 95%, 96%, 97%, 98% identity with SEQ ID No 21.

In a second aspect, the present invention relates to a recombinant vector comprising at least an isolated nucleic acid sequence according to the invention. More particularly, the present invention relates to a recombinant viral vector comprising at least an isolated nucleic acid sequence according to the invention

The present invention relates to a recombinant viral vector comprising at least:
I. a nucleotide according to the invention, and
II. a control element, in particular a muscle-specific control element, operably linked to and driving the expression of said polynucleotide, wherein said control element is preferably a promoter.

In vivo gene therapy is a direct method of inserting the genetic material into the targeted tissue, and transduction takes place within the transfected cells. A gene therapy vector according to the invention is desirably delivered locally, or systematically.

Among possible gene therapy vectors appropriate for the invention, viral vectors are particularly indicated. More particularly, the present invention relates to a recombinant viral vector comprising at least an isolated nucleic acid sequence according to the invention, for gene therapy.

Adeno-associated virus (AAV) is a replication-deficient parvovirus, the single stranded DNA genome of which is about 4.7 kb in length, including 145 nucleotides inverted terminal repeat (ITR).

AAV possesses many features that make it attractive for delivering foreign DNA to cells, in particular for gene therapy. Multiple studies have demonstrated long-term recombinant AAV-mediated protein expression in muscle and that the muscle is capable of stable expression of secreted protein therapeutics.

As known by a person skilled in the art of designing and manipulating vectors for gene therapy, and particularly AAV, grows only in cells in which certain functions are provided by a co-infecting helper virus.

Recombinant AAV genomes of the invention comprises nucleic acid molecules of the invention and one or more AAV ITRs flanking a nucleic acid molecule.

Reported clinical doses for AAV-based viral vectors range from 10¹¹ to 10¹⁴ vector genomes per patient, depending on therapeutic area.

In a particular embodiment, a recombinant vector of the invention is a viral vector. In a more particular embodiment, a recombinant vector of the invention is a recombinant AAV (rAAV) vector or a recombinant lentiviral vector.

In a more particular embodiment, the vector is of the serotype AAV1, AAV2, AAV3, AAV4, AAV5, AAV6, AAV7, AAV8, AAV9, AAV10, AAV11, AAV12 or AAV13 serotype, or a derivative of a known serotype. In certain embodiment, the vector exhibits a desired tissue specific or tropism or other desirable properties for a pharmaceutical composition or gene therapy for various indications.

In another particular embodiment, the muscle-specific control element is human skeletal actin gene element, a human myosin kinase promoter or a synthetic muscle specific promoter C5.12.

In certain embodiments, a vector of the invention comprises a polyadenylation (polyA) sequence for inserting a polyA sequence into a transcribed mRNA.

In a third aspect, the present invention relates to a composition comprising a recombinant vector according to the invention and a therapeutically acceptable carrier, diluent or excipient.

In a forth aspect, the present invention relates to a recombinant vector comprising at least an isolated nucleic acid sequence according to the invention or a composition comprising a recombinant vector according to the invention, for its use as a medicament.

More particularly, the present invention relates to a recombinant vector comprising at least an isolated nucleic acid sequence according to the invention or a composition comprising a recombinant vector according to the invention, for its use as a medicament for the treatment of a dysferlinopathy.

Vectors suitable for the invention can be produced by using any the methods known in the art. Viral particles can be produced by using any the methods known in the art such as using stable mammalian cell lines.

Another aspect of the invention provides a method of producing a viral vector of the invention, comprising culturing a cell that has been transfected with a viral vector of the invention and recovering the viral particles from the supernatant of the transfected cells.

Another aspect of the invention provides viral particles comprising any of the viral vectors of the invention.

Another aspect of the invention provides a composition comprising a vector according to the invention.

According to an embodiment, a composition according to the invention is a pharmaceutical composition further comprising a therapeutically compatible carrier, diluent or excipient.

For administration, effective amounts and therapeutically effective amounts, also referred as doses, may be initially estimated based on results from in vitro assays and/or animal model studies. A vector according to the present invention, when injected to a mouse model, such as for example a deficient-dysferlin mouse model (Dysf Y1159X/Y1159X), leads to the production of a recombinant protein; analysis of expressed protein and the quantification of dystrophic features lead to the characterization of the polypeptide encoded by said vector according to the invention.

The actual dose of vector according to the invention will vary according to the particular vector used, the mode of administration, the treatment goal, the individual and the cell types being targeted, and may be determined by methods standard in the art. The actual dose of vector according to the invention may also be determined by a physician taking into account physical and physiological factors, severity of condition and/or route of administration. Exemplary doses may range from about 1×10¹⁰ to about 1×10¹⁵ vector genomes per kilogram of body weight.

According to an embodiment, a pharmaceutical composition according to the invention is in a dosage form comprising at least 5×10¹¹ vector genomes, more preferably from about 5×10¹¹ to about 10¹⁵ vector genomes.

The present invention also provides a vector according to the invention, or a composition according to the invention, for its use as a medicament.

The present invention also provides a vector according to the invention, or a composition according to the invention, for its use as a medicament for treating dysferlinopathy.

Even more particularly, the present invention relates to a recombinant vector comprising at least an isolated nucleic acid sequence according to the invention or a composition comprising a recombinant vector according to the invention, for its use as a medicament for the treatment of a dysferlinopathy chosen among Miyoshi myopathy (MM), limb girdle muscular dystrophy (LGMD) and limb girdle muscular dystrophy type 2B (LGMD2B or LGMDR2).

According to an embodiment, the present invention provides a vector according to the invention, or a composition according to the invention, for its use as a medicament for treating Miyoshi myopathy (MM) or limb girdle muscular dystrophy type 2B (LGMD2B or LGMDR2).

In a particular embodiment, a vector according to the invention or a composition is in a form suitable for intramuscular injection, intravenous injection, parental delivery or systemic administration.

The present invention also provides a method for the treatment of dysferlinopathy in a subject in need thereof, the method comprising administering to the subject a therapeutically effective amount of a vector according to the invention or a composition according to the invention.

The following examples illustrates, without limiting it, the key role played by a polynucleotide encoding for exon 40a in cellular processes of dysferlinopathy, supporting the medical solution provided by a gene therapy vector according to the invention and its use for treating dysferlinopathy.

Any one embodiment described herein can be combined with any one or more other embodiments of the invention, unless such combination is expressly disclaimed or improper.

Characteristics, advantages and uses of the subject-matter of the present invention are more detailed hereunder, in an illustrated and non-limiting way. When present, the disclosure of the ranges expressed as “from . . . to . . . ” mean that the limits are included in said ranges.

### EXAMPLES

### Example 1: Characterization of the Role of Alternative Exon 40a of Dysferlin

### 1. Material and Methods

### Cell Cultures

WT (C25) and DYSF-null (AB320) human myoblasts cells lines were kindly given by Vincent Mouly from the Myology Research Center (Paris, France). Cells were grown in a humidified environment at 37° C. and 5% CO₂ in Dulbecco's modified Eagle's medium, supplemented with 15% medium 199, 15% fetal bovine serum, 25 μg/mL fetuin, 5 ng/ml hEGF, 0.5 ng/mL bFGF, 5 μg/mL insulin and 0.2 μg/mL dexamethasone.

C2C12 murine myoblasts and HEK cells lines were bought at ATCC. Cells were grown in a humidified environment at 37° C. and 5% CO₂ in Dulbecco's modified Eagle's medium (ThermoFisher), supplemented with 20% fetal bovine serum and 100 μg/mL antibiotic antimycotic (GE Healthcare, ref P11-002).

### Plasmid Construction

GFP-dysferlin1 plasmid was a generous gift from Dr. Kate Bushby, it contains the transcript of the main isoform of dysferlin (exon 1 to 55). mCherry-dysferlin1 plasmid was generated by GFP switching to mCherry (Shaner et al, Nature Biotech., 21 Nov. 2004) using the EcoRI and KpnI restriction enzymatic sites.

Coding sequence of GFP-dysferlin11 was generated by Cliniscience by inserting the alternative exon 40a between exon 40 and exon 41 into the GFP-dysferlin1 plasmid to reproduce the transcript 11 of the dysferlin. mCherry-dysferlin11 plasmid was generated by GFP switching to mCherry using the EcoRI and KpnI restriction enzymatic sites. Dysferlin11 constructs to identify the cleavage site by calpain in DYSF exon 40a were created by PCR fusion. Table 1 summarizes the primers used to create dysferlin11 constructs by PCR fusion.

TABLE 1

| | | |
|---|---|---|
| pcDNA Dysf11 | Forward | GCCCCTCATCCCCATCCAGAACACG |
| delN | | GCTTCTCCTCCATCCAGTCCTC |
| | Reverse | GATGGAGGAGAAGCCGTGTTCTGGA |
| | | TGGGGATGAGGGGCTCCTTGTCATC |
| pcDNA Dysf11 | Forward | GTCGAGCTTGGCCCCCACTGAGGAA |
| delC | | GAGTTCATCGATTGGTGGAGC |
| | Reverse | CCAATCGATGAACTCTTCCTCAGTG |
| | | GGGGCCAAGCTCGACAGACCGTC |
| pcDNA Dysf11 | Forward | CCTCATCCCCATCCAGAGCTTGGCC |
| delN1 | | CCCACTAACACGGCTTCTCC |
| | Reverse | GTTAGTGGGGGCCAAGCTCTGGATG |
| | | GGGATGAGGGGCTCCTTGTCATC |
| pcDNA Dysf11 | Forward | GCTTGCAGACGGTCTGTCGAACACG |
| delN2 | | GCTTCTCCTCCATCCAGTCC |
| | Reverse | GGAGGAGAAGCCGTGTTCGACAGAC |
| | | CGTCTGCAAGCTGGATGGGG |

### Cell Scraping Injury, SDS-PAGE and Western Blot

HEK cells were plated at 70% confluence in 6-well plate (VWR) and transfected using Lipofectamine 2000 (Invitrogen) per manufacturer's directions. After 24 h, cells were injured with 30 μM ionomycin and cell scrappers, then they were pelleted at 300 g for 5 min and the cell pellet was solubilized in RIPA buffer (Life technologies) and protease inhibitor cocktail (Life technologies). Samples were separated by SDS-PAGE on 3-8% NuPAGE Tris-Acetate gels (Life technologies) using Chameleon Duo as a size marker and transferred onto nitrocellulose membranes (at 100V for 3 h at 4° C.). Membranes were blocked using fluorescent WB blocking buffer (tebu-bio) in TBS 1× for 1 hour at room temperature. Primary antibodies (Hamlet, 1:150, abcam 75571) were then diluted in blocking buffer and incubated overnight at 4° C. After washing in TBS-T, membranes were then incubated with secondary antibodies (IRDye 800CW Donkey anti-mouse, Li-Cor, ref 926-32212), which were diluted 1:10000 in blocking buffer for 45 min at room temperature. The membranes were washed in TBS-T and developed using NIR Fluorescence LI-COR. Actin (1:5000, Merck MA1501r) was detected using a dilution of 1:10000 of secondary antibodies (IRDye 680RD Donkey anti-mouse, Li-Cor, ref 925-68072).

### Immunofluorescence

C2C12 myoblasts were grown on Lab-TEK II™ (Fisher Scientific) and transfected using Lipofectamine 2000 (Invitrogen) per manufacturer's directions. After 24 h, cells were injured with glass beads, fixed with 4% paraformaldehyde for 10 minutes and then washed in PBS. Cells were then incubated for 10 minutes with a permeabilization solution (200 μL of PBS 1×+0.5% triton X-100+protease inhibitors cocktail) (Roche). From there, cells were exposed to a blocking buffer (PBS+1% BSA+protease inhibitors cocktail) for 30 minutes. The primary antibody (Hamlet, 1:40, abcam 75571) was applied in blocking buffer for 3 hours at room temperature, followed by a wash in PBS and 1 hour of contact with the secondary antibody (Dylight 550 donkey anti mouse IgG, 1:100, abcam 96876) in blocking buffer. After washing in PBS, Lab-TEK II™ were mounted with Vectashield-Dapi 25 ng/ml and kept at 4° C. until pictures were taken. Observation was performed using a Zeiss Axio Imager Z2 microscope (40× objective), and images were processed with ZEN software and/or ImageJ software.

### Membrane Repair Assay

WT (C25) and DYSF-null (AB320) human myoblasts were plated at 50% confluence on 6-well plate (VWR) and transfected using Lipofectamine 2000 (Invitrogen) per manufacturer's directions. After 24 h, cells were visualized in the presence of Ca²⁺ (1 mM) and membrane-impermeable dye FM 1-43 (2.5 μM, Molecular Probes) with a confocal microscope (LSM 800, 63× objective, ZEISS). Membrane damage was applied to myoblast using a UV laser to irradiate a 0.33 μm² area at maximum power for 15 seconds at t=10 sec. Images were captured every second for 5 min, and the mean fluorescence intensity of FM1-43 was measured on a 13.5 μm² area around the damage with the Zeiss LSM 800 imaging software.

### Osmotic Shock Assay

WT (C25) and DYSF-null (AB320) human myoblasts were plated at 70% confluence on 12-well plate (VWR) and transfected using Lipofectamine 2000 (Invitrogen) per manufacturer's directions. After 24 h, myoblasts were washed with distilled water and hypo-osmotic shock was performed by incubating cells with distilled water. Cell fluorescence was followed with Fast calcium imaging observer (Axio observer. Z1/7, 10× objective, ZEISS). Images were captured every minute for 30 minutes.

### Transferrin Uptakes and Recycling Assays

WT (C25) and DYSF-null (AB320) human myoblasts were plated at 70% confluence on Lab-TEK II™ (Fisher Scientific) and transfected using Lipofectamine 2000 (Invitrogen) per manufacturer's directions. After 24 h, myoblasts were incubated in Dulbecco's modified Eagle's medium, supplemented with 1% L-glutamine and 0.5% bovine serum albumin at 37° C. and 5% CO₂ for 30 minutes. Then, cells were incubated in the same medium with 25 μg/mL transferrin from human serum (coupled with Alexa Fluor 488, Thermo Scientific) at 37° C. and 5% CO₂ for 30 minutes. For transferrin recycling assays, cells were washed in cold PBS and incubated in Dulbecco's modified Eagle's medium, supplemented with 15% medium 199, 15% fetal bovine serum, 25 μg/mL fetuin, 5 ng/ml hEGF, 0.5 ng/ml bFGF, 5 μg/mL insulin and 0.2 μg/mL dexamethasone. Then, myoblasts were washed in PBS and incubated in stripping buffer (NaCl, acid acetic) during a few seconds. After that, cells were washed in PBS, fixed with 4% paraformaldehyde for 10 minutes and washed again in PBS. Lab-TEK II™ were mounted with Vectashield-Dapi 25 ng/ml and kept at 4° C. until pictures were taken. Observation was performed using a Zeiss Axio Imager Z2 microscope (20× objective), and images were processed with ZEN software and/or ImageJ software.

### Statistical Analysis

Individual means were compared using the non-parametric Mann-Whitney test. The power of the tests was strictly superior at 92%. Differences were considered to be statistically significant if p<0.05 (*) or very significant if p<0.01 (**).

### 2. Results

Dysferlinopathy encompasses muscular dystrophies caused by mutations in the DYSF gene. Dysferlin gene encodes a sarcolemmal protein required for repairing muscle cell damage. It consists of calcium-dependent lipid binding domains and a transmembrane domain. Dysferlin-deficient muscle fibers have a defect in membrane repair.

### 2.1. Cleavage Site by Calpain in DYSF Exon 40a

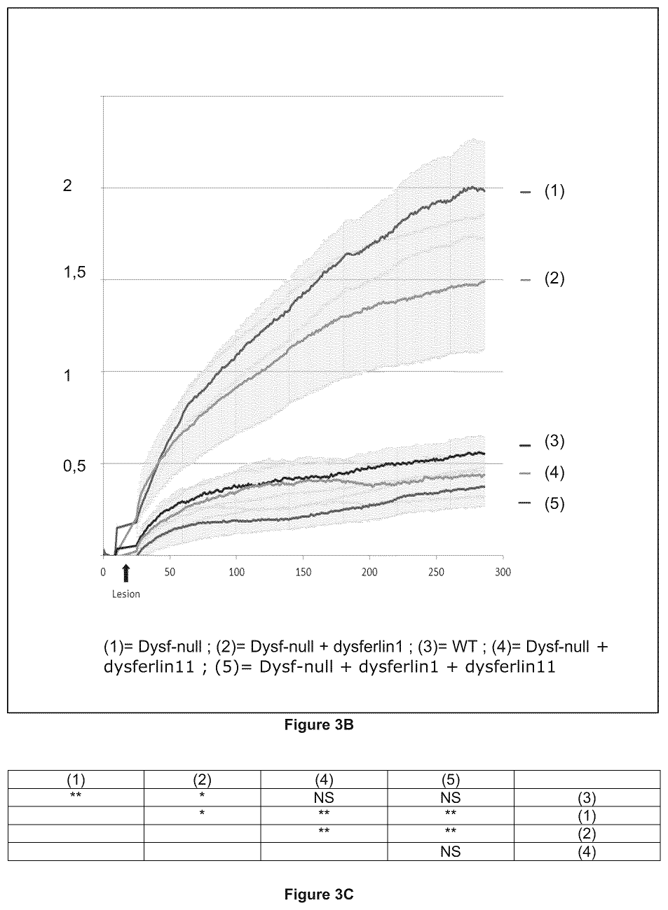

Several publications demonstrated that membrane injury causes calcium influx at injury sites which induces local activation of calpains. This activation led to dysferlin cleavage by calpains. In 2014, it has been shown that the cleavage site by calpain resides in the DYSF exon 40a. To identify the exact location of cleavage site by calpain in dysferlin exon 40a, four plasmids containing the dysferlin transcript 11 with deletions of fragments in exon 40a were constructed: Dysf11deIN, Dysf11delC, Dysf11 delN1, Dysf11deIN2 (FIG. 3A). Injury assays by ionomycin and cell scrappers were performed in HEK cells and followed dysferlin cleavage by western blot analysis with Hamlet-1 antibody (FIG. 3B). Protein extracted from injured cells transfected with dysferlin transcript 11 revealed the presence of the mini-dysferlin band as the expected size whereas in injured HEK transfected with dysferlin transcript 1 no signal for the mini-dysferlin was detected.

When exon 40a is deleted in two parts (one on the N-terminal DeIN side and one on the C-terminal DelC side), deletion of the N-terminal part had a hardly effect on the intensity of the band corresponding to the mini-dysferlin. Thus, the N-terminal part would be more important than the C-terminal part for the recognition and cleavage of this region by calpains.

Mutations and small deletions were performed in DYSF exon 40a. Surprisingly, none of the mutations tested abolishes the cleavage of this domain by calpains, as represented in Table 2.

TABLE 2

| | | Calpain |
|---|---|---|
| ID | Exon 40a modification | cleavage |
| S1485A | CTTGCAGACGGTCTGTCGAGC | Yes |
| P1487A | TTGGCCCCCACTAACACGGCT | |
| | GCTCCTGCATCCAGTCCTCAT | |
| S1485A | CTTGCAGACGGTCTGTCGAGC | Yes |
| P1487A | TTGGCCCCCACTAACACGGCT | |
| S1489G | GCTCCTGCATCCGGTCCTCAT | |
| S1476Y | CTTGCAGACGGTCTGTACAGC | Yes |
| | TTGGCGGGCACTAACACGGCT | |
| | TCTCCTCCATCCAGTCCTCAT | |
| S1476A | CTTGCAGACGGTCTGGCGGGC | Yes |
| S1477G | TTGGCCCCCACTAACACGGCT | |
| | TCTCCTCCATCCAGTCCTCAT | |
| T1481A | CTTGCAGACGGTCTGTCGAGC | Yes |
| | TTGGCCCCCGCTAACACGGCT | |
| | TCTCCTCCATCCAGTCCTCAT | |
| T1483K | CTTGCAGACGGTCTGTCGAGC | Yes |
| | TTGGCCCCCACTAACAAGGCT | |
| | TCTCCTCCATCCAGTCCTCAT | |
| S1488Y | CTTGCAGACGGTCTGTCGAGC | Yes |
| | TTGGCCCCCACTAACACGGCT | |
| | TCTCCTCCAACCAGTCCTCAT | |
| Del PTNT | CTTGCAGACGGTCTGTCGAGC | Yes |
| | TTGGCC------------GCT | |
| | TCTCCTCCATCCAGTCCTCAT | |
| Del P1480 | CTTGCAGACGGTCTGTCGAGC | Yes |
| | TTGGCC---ACTAACACGGCT | |
| | TCTCCTCCATCCAGTCCTCAT | |
| P14805 | CTTGCAGACGGTCTGTCGAGC | Yes |
| | TTGGCCCTCACTAACACGGCT | |
| | TCTCCTCCATCCAGTCCTCAT | |
| A1472Y | CTTTATGACGGTCTGTCGAGC | Yes |
| | TTGGCCCCCACTAACACGGCT | |
| | TCTCCTCCATCCAGTCCTCAT | |
| Del LAD | ---------GGTCTGTCGAGC | Yes |
| | TTGGCCCCCACTAACACGGCT | |
| | TCTCCTCCATCCAGTCCTCAT | |

These results suggest that calpains recognize and cleave a structural domain instead of a precise amino acids sequence. For instance, injured HEK cells transfected with dysferlin transcript 11 delN1 (deletion of exon 40a first seven aa) and transcript 11 delN2 (deletion of exon 40a second quarter) present a weaker mini-dysferlin band compared to injured HEK cells transfected with dysferlin transcript 11 (FIG. 3B). Inhibitor of calpain 1 and 2 prevents calpain cleavage in DYSF exon 40a (data not shown), confirming that cleavage is due to calpain activity. Altogether these experiments demonstrate that dysferlin is cleaved in exon40a by calpains (1 and 2) in the central region of this sequence. Furthermore, the deletions made within exon 40a indicate that calpains recognize a sequence towards the N-terminal part of the exon. This minimal sequence is composed of the first 11 amino acids of the polypeptide encoded by exon 40a.

### 2.2. Dysferlin Localization in Muscle Cell

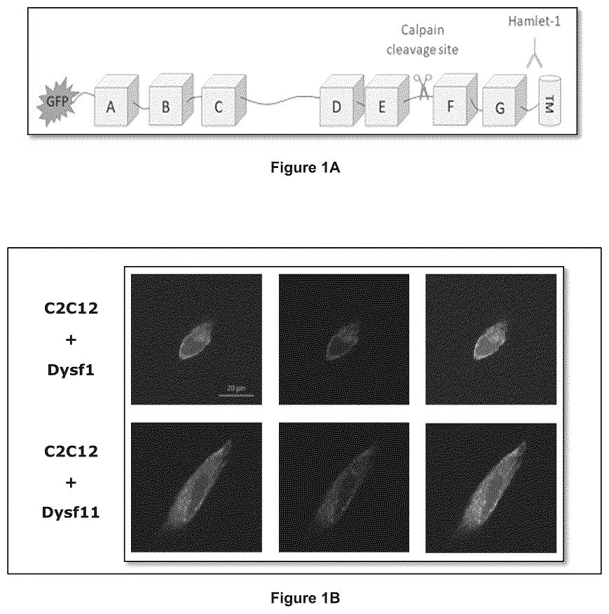

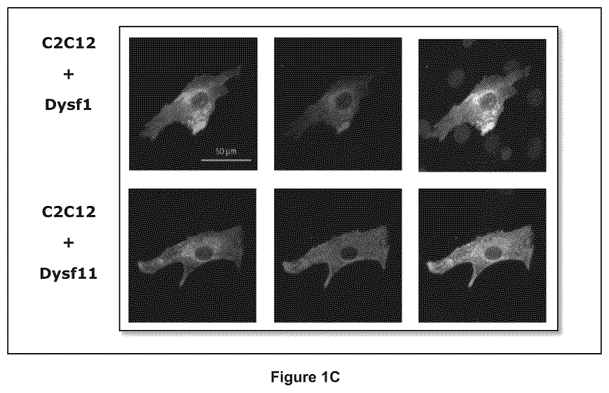

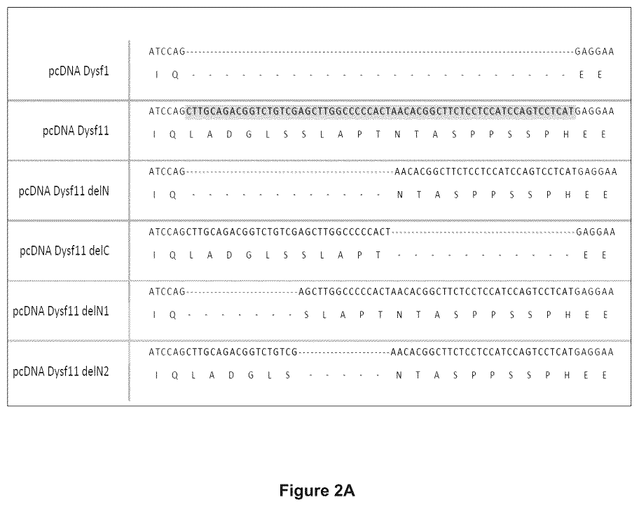

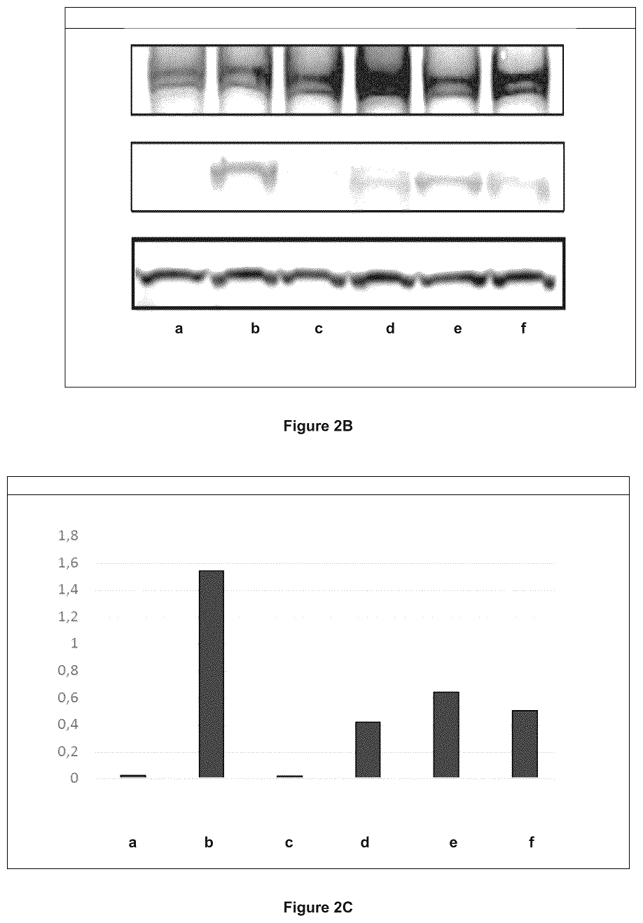

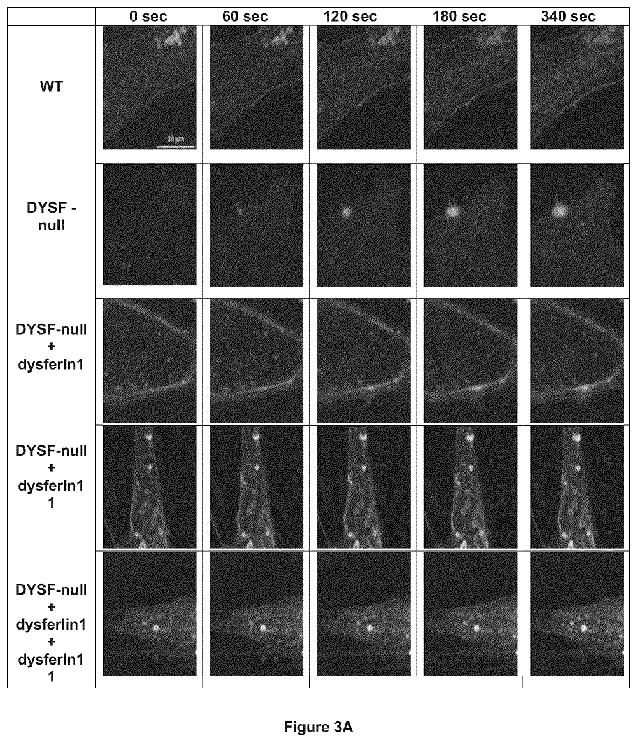

In 2013, Lek et al (“Calpains, cleaved Mini-DysferlinC72 and L-type channels underpin calcium-dependent muscle membrane repair”, J. Neurosc., Mar. 20, 2013, 33(12):5085-94) demonstrated that during membrane repair process, dysferlin cleavage by calpains within exon 40a releasing a C-terminal fragment named mini-dysferlin which is enriched at membrane injury sites. To explore the fate of this mini-dysferlin, murine myoblasts were transfected with dysferlin transcript 1 (major transcript) or dysferlin transcript 11 (transcript containing exon 40a) to monitor the dysferlin localization after membrane injury (FIGS. 1B and 1C). Lesions were generated by glass beads injuries. Dysferlin labelling was done using GFP staining for the N-terminal domain and Hamlet-1 antibody recognizing a C-terminal epitope. In uninjured myoblasts transfected with dysferlin transcript 1 or dysferlin transcript 11, no difference in localization between the N-terminal and C-terminal parts of dysferlin were detected (FIG. 1B). In injured myoblasts transfected with dysferlin transcript1, the same co-localization of the N and C-terminal parts were observed (FIG. 1C). In contrast to what was observed, in injured myoblasts transfected with dysferlin transcript 11, the C-terminal part of dysferlin revealed by the Hamlet-1 antibody was clearly enriched at the membrane level and was no longer uniformly co-located with the N-terminal part (FIG. 1C). This result supports the fact that when cleaved the C-terminal fragment of dysferlin is recruited to membrane injury site.

### 2.3. Dysferlin Transcript 11 is Essential in Muscle Cell Membrane Repair

To demonstrate the importance of the dysferlin transcript 11, a membrane repair assay was performed on wild-type human myoblasts (WT), dysferlin-null human myoblasts (DYSF-null) and transfected dysferlin-null human myoblasts (DYSF-null+mCherry-dysferlin1, DYSF-null+mCherry-dysferlin11 and DYSF-null+mCherry-dysferlin1+mCherry-dysferlin11) in presence of calcium and membrane-impermeable dye FM 1-43 (FIG. 3A). Laser membrane injuries on wild-type myoblasts lead to a FM dye entry that cease within a minute of injury whereas it continued steadily after 5 min in dysferlin-null myoblasts. When dysferlin transcript 1 is expressed in dysferlin-null myoblasts, we can see an important FM dye entry during injury, nevertheless it is slowing down after a few minutes. On the other hand, dysferlin-null myoblasts expressing dysferlin transcript 11 that are injured show a slight FM dye entry that cease within a minute, such as wild-type myoblasts. A significative difference (p<0.01) was observed between un-transfected dysferlin-null myoblasts and dysferlin-null myoblasts expressing dysferlin transcript 11. These results indicate that dysferlin transcript 11 is essential in the muscle cell membrane repair mechanism after laser-induced injury.

2.4. Dysferlin Transcript 11 Participates in the Protection of Membrane Myoblasts from Mechanical Stress Induced by Osmotic Shock

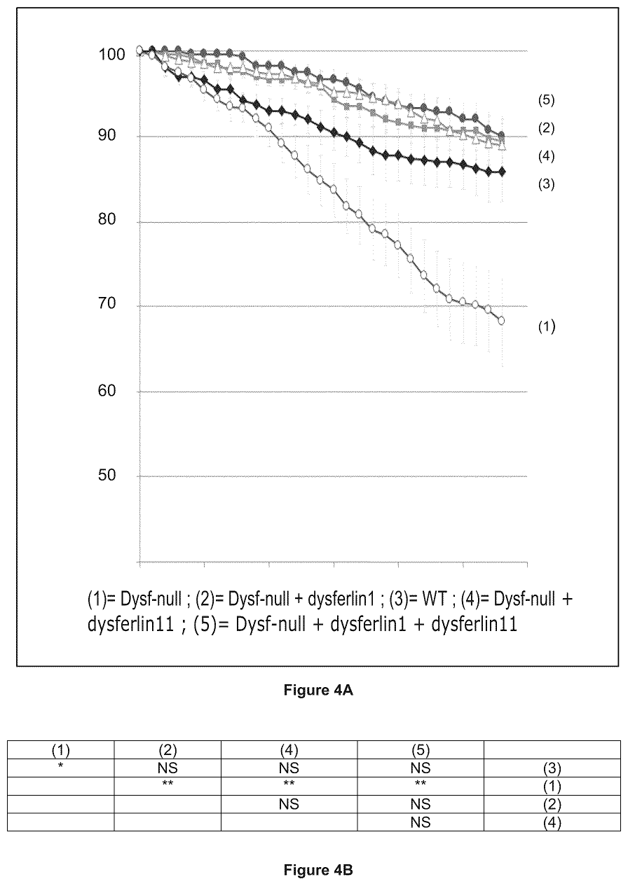

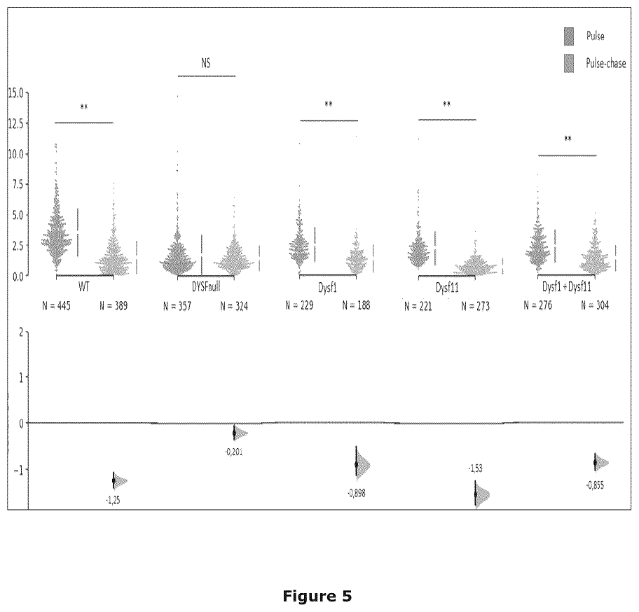

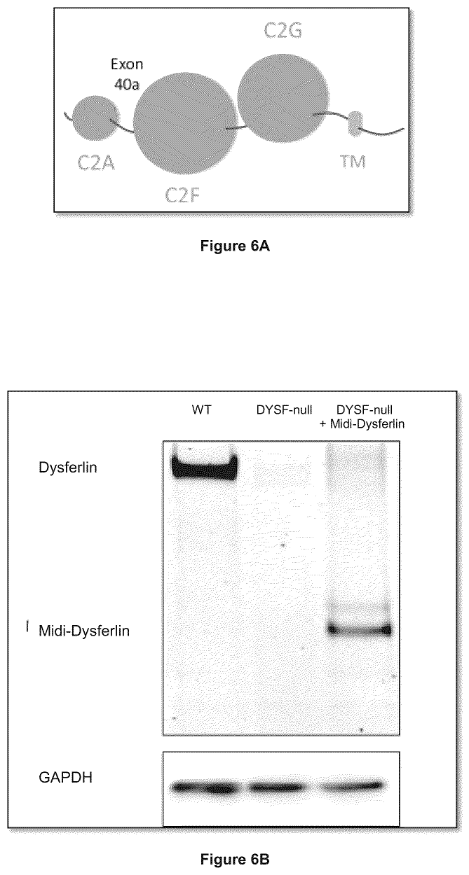

The role of dysferlin transcript 11 in the protection of myoblast membrane was explored by an osmotic shock assay on wild-type human myoblasts (WT), dysferlin-null human myoblasts (DYSF-null) and transfected dysferlin-null human myoblasts (DYSF-null+GFP-dysferlin1, DYSF-null+GFP-dysferlin11 and DYSF-null+GFP-dysferlin1+GFP-dysferlin11) (FIG. 4A). Cells were placed in a hypotonic solution. This hypo-osmotic shock leads to cell swelling which induces mechanical stresses on the cell membrane. To monitor the integrity of the cell membrane, we have observed the plasmids GFP fluorescence that leaks out of the cells when the plasma membrane is damaged. The percentage of cell survival for wild-type myoblasts during hypo-osmotic shock decreased slightly the first fifteen minutes but it seems to stabilize after that (86% of cell survival percentage after 20 min of hypo-osmotic shock). On the contrary, hypo-osmotic shock on dysferlin-null myoblasts induces a decrease of cell survival percentage not stabilized after 20 min (68% of cell survival percentage after 20 min of hypo-osmotic shock). When dysferlin transcript 1 or dysferlin transcript 11 was expressed in dysferlin-null myoblasts, we detected a slight decrease of cell survival percentage during hypo-osmotic shock, similar to wild-type myoblasts (90% of cell survival percentage after 20 min of hypo-osmotic shock). These observations showed that dysferlin transcript 1 and dysferlin transcript 11 may protect myoblasts from mechanical stresses induced by hypo-osmotic shocks.

### 2.5. Dysferlin Transcript 11 Participates in Protein Vesicle Trafficking

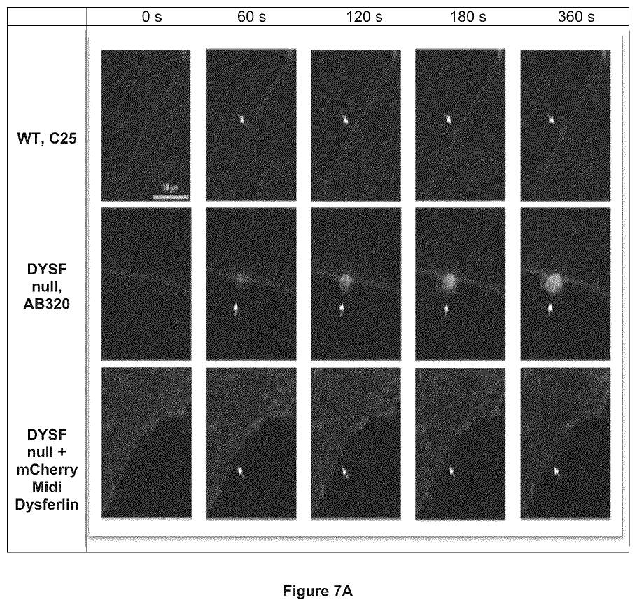

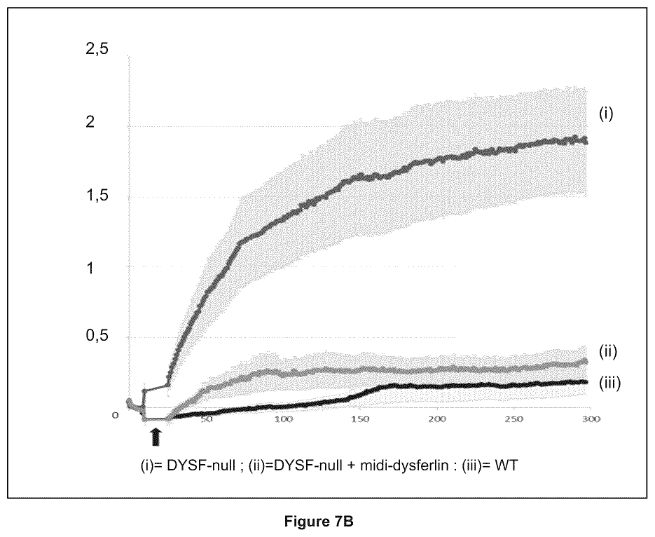

Several publications have involved dysferlin in protein vesicle trafficking. To explore the role of dysferlin transcript 11 in protein vesicle trafficking, a transferrin assay was performed on wild-type human myoblasts (WT), dysferlin-null human myoblasts (DYSF-null) and transfected dysferlin-null human myoblasts (DYSF-null+mCherry-dysferlin1, DYSF-null+mCherry-dysferlin11 and DYSF-null+mCherry-dysferlin1+mCherry-dysferlin11) (FIG. 7). Transferrin is a molecule rapidly internalized into cells via the transferrin receptor. After internalization, transferrin is recycled to the plasma membrane. To explore the internalization process, myoblasts were incubated for 30 min with transferrin coupled to the fluorophore Alexa-488. The recycling function was also evaluated by incubating myoblasts for 30 min with transferrin and then incubating myoblasts for 30 min in transferrin-free medium. For each cell, we quantified the intensity of transferrin. Internalization function (pulse) compared with recycling function (pulse-chase) showed a significative difference for wild-type myoblasts compared to dysferlin-null myoblasts. The difference between fluorescence levels for pulse and pulse-chase were greater when comparing control myoblasts (−1.25 effect size, Cohen's coefficient) to dysferlin-deficient myoblasts (−0.2 effect size). In dysferlin-null myoblasts expressing dysferlin transcript 1 or dysferlin transcript 11, significant difference between pulse and pulse-chase were observed (−0.898 effect size for dysferlin transcript 1 and −1.53 effect size for dysferlin transcript 11). These observations indicated that both transcripts participated in transferrin trafficking in muscle cell.

This study demonstrates the importance of the dysferlin11 transcript which contains the alternative exon 40a. Indeed, membrane injuries of muscle cells induce the establishment of a repair mechanism that causes dysferlin cleavage by calpains within exon 40a, releasing a C-terminal fragment named mini-dysferlin.

Cell scrape injury and western blot analysis performed in this study show that cleavage in DYSF exon 40a is carried out by calpains 1 and/or 2. Several studies expose the predictions of calpains cleavage, but there seems to be no explicit rule for calpain specificity. Actually, amino acid sequences around cleavage sites by calpain are very diverse. It is therefore difficult to precisely determine the location of cleavage sites by calpain. Our results demonstrate that partial deletion of exon 40a decrease calpain cleavage (delC, delN1 and delN2) and deletion of exon 40a first half prevent calpain cleavage (delN). Therefore, the entire sequence of exon 40a is important for the cleavage of dysferlin by calpains but N-terminal part of this alternative exon is more important than others.

Results show that substitutions and small deletions in DYSF exon 40a do not prevent calpain cleavage. Thus, it has been observed that amino acid substitution or deletion of one to seven amino acids do not prevent dysferlin cleavage by calpains. Only the deletion of the twelve first amino acids of DYSF exon 40a avoids cleavage of dysferlin. These observations confirm that calpains recognize a large amino acid sequence to cleave dysferlin.

On the other hand, myoferlin, which is a member of ferlin protein family, is also cleaved by calpains within an alternative exon, releasing a C-terminal fragment that has 62.82% amino acid identity with the mini-dysferlin. There seems to be an evolutionary preservation of enzymatic cleavage of both dysferlin and myoferlin. Indeed, DYSF alternative exon 40a is conserved in the majority of mammals.

Given the importance of the region coded by DYSF exon 40a (site of dysferlin cleavage by calpains during repair of the muscle cell membrane), dysferlin transcript 11 must have a particular importance in the functions of the muscle cell. Actually, our results indicate that this transcript is essential to the reparation of the muscle cell membrane. Main transcript of dysferlin (transcript 1) seems to be little involved in the repair mechanism of the muscle cell membrane when the latter suffer a laser-injury. In that case, only dysferlin transcript 11 which contains exon 40a makes it possible to repair the muscle cell membrane injured with a laser. These results are in line with the work of Lek and her team in 2013 that showed dysferlin cleavage at exon 40a by calpains and accumulation of mini-dysferlin at lesion site during repair of the muscle cell membrane.

Otherwise, dysferlin is involved in other muscle cell functions. Our results confirm this previous work: dysferlin-null myoblasts are more likely to die from hypo-osmotic shock unlike wild-type myoblasts. Our results also show that restoration of dysferlin transcript 1 and/or dysferlin transcript 11 seem to increase the cells resistance to mechanical stress induced by osmotic shock. This observation indicates that alternative exon 40a is not essential for muscle cell membrane protection, but dysferlin transcript 11 is able to accomplish it.

Moreover, dysferlin is involved in protein vesicle trafficking. It is known that dysferlin-null myoblasts accumulate transferrin and transferrin endocytic recycling is delayed in these cells. The present data show equally an abnormal vesicular trafficking in dysferlin-null myoblasts compared to wild-type myoblasts and dysferlin-null myoblasts with a dysferlin restoration. These data demonstrate that dysferlin-null myoblasts have a lower transferrin accumulation than wild-type myoblasts, and that they do not show a significant difference between endocytosis function (pulse) and recycling function (pulse-chase). When dysferlin transcript 1 and/or dysferlin transcript 11 is restored in dysferlin-null myoblasts, we show again a significant difference between endocytosis and recycling functions which demonstrates a restoration of vesicular trafficking. These two dysferlin transcripts seem to participate in transferrin trafficking in muscle cell. This observation indicates, as before, that alternative exon 40a is not essential for protein vesicle trafficking, but dysferlin transcript 11 is able to accomplish it.

All of these results indicate that dysferlin transcript 1 but also dysferlin transcript 11 have an important place in the functions of the muscle cell. In terms of therapy, these findings suggest that dysferlin transcript containing exon 40a should be restored in patients with dysferlinopathy. Currently, all therapeutic studies conducted so far restore only the main transcript of dysferlin (transcript 1). These studies show promising results: high levels of dysferlin expression, improvement of the histological aspect of the muscle, reduction of necrotic fibers and global improvement in locomotor activity.

Regarding the muscle membrane repair function, the improvements obtained do not restore the function to the same level as seen in the wild-type individuals. It would be interesting to restore dysferlin transcript 11 in models of dysferlinopathies to obtain better results in the repair function of the muscle cell membrane.

Among these SNPs, only two missense polymorphisms were predicted pathogenic by UMD-Predictor: c.4704C>T and c.4707G>T. Therefore, we carried out constructions in which we induced a substitution of the amino acids concerned by these two SNPs predicted pathogenic. Cell scrape injury and western blot analysis performed with these two constructions show that amino acid substitutions at these positions do not avoid the cleavage of dysferlin by calpains during the repair mechanism of muscle cell membrane. Moreover, our results indicate that substitutions and small deletions in DYSF exon 40a do not prevent calpain cleavage. These observations indicate that variants in exon 40a do not prevent the formation of mini-dysferlin, which has a major role in membrane repair mechanism. Knowing this, we can assume that variants in the alternative exon 40a do not lead to membrane repair defect. Nevertheless, as transcript containing exon 40a are strongly expressed in liver, lung, placenta and pancreas (between 40 and 60%), variants present in this exon could lead to non-muscular deleterious phenotype.

These data demonstrate the importance of the dysferlin transcript 11, which contains the alternative exon 40a, in muscle cell functions and more particularly in the reparation mechanism of the muscle cell membrane. During this mechanism, calpains 1 and/or 2 cleave dysferlin in the first part of DYSF exon 40a, releasing a C-terminal fragment named mini-dysferlin. Our results show that dysferlin transcript 11 is essential in muscle cell membrane repair, but it also participates in muscle cell membrane protection and protein vesicle trafficking. All these results indicate that dysferlin transcript 11 have an important place in the functions of the muscle cell. In terms of therapy, these findings suggest that dysferlin transcript containing exon 40a should be restore in patients with dysferlinopathy.

### Example 2: Midi-Dysferlin for Gene Therapy

The inventors designed a midi-dysferlin protein, comprising at least, from its Nt extremity to its Ct extremity:
A C2A domain, or a functional equivalent thereof
A polypeptide encoded by exon40a, a functional fragment thereof or a functional derivative thereof
A C2F domain, or a functional equivalent thereof
A C2G domain, or a functional equivalent thereof
A transmembrane domain (TM).

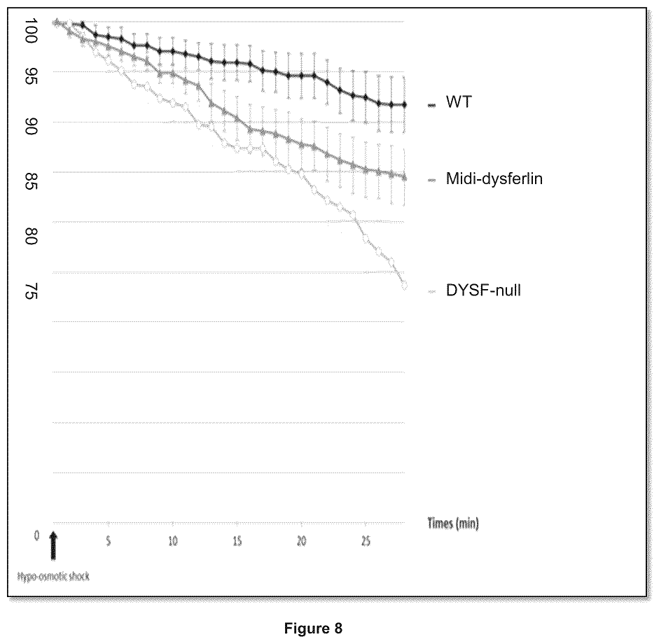

Midi-dysferlin (SEQ ID No 21) was transfected into dysferlin-null myoblasts. Western blot performed with proteins from wild-type myoblasts (WT, C25), dysferlin-null myoblasts (DYSF-null, AB320) and transfected dysferlin-null myoblasts (DYSF-null+Midi-dysferlin), wherein Hamlet antibodies were used to detect dysferlin and midi-dysferlin, showed the expression of midi-dysferlin in transfected dysferlin-null myoblasts (FIGS. 8A and 8B).

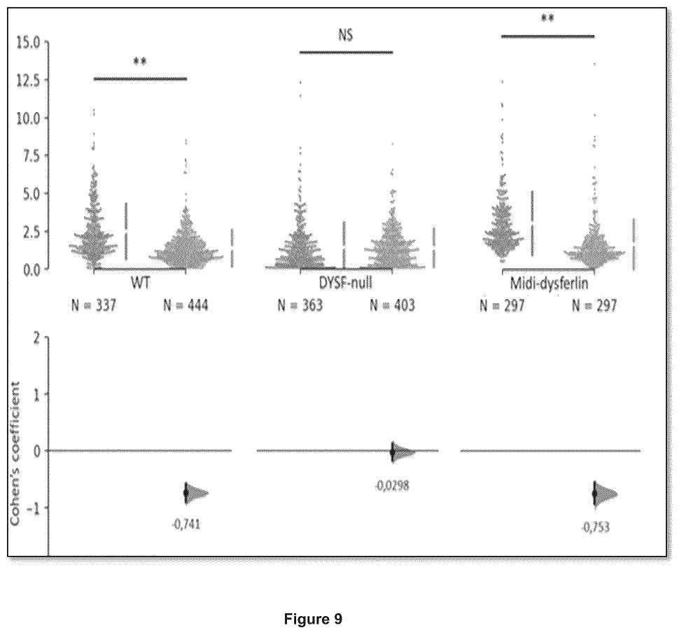

Experimental data with midi-dysferlin, which were performed in conditions similar to those described in example 1 with dysferlin, show that:
Midi-dysferlin allows a fully complete restoration of muscle-cell membrane repair equivalent to what is observed in control cells. (FIGS. 7A and 7B).
Midi-dysferlin allows a partial restoration of myoblasts membrane protection from mechanical stress induced by osmotic shock (FIG. 8).
Midi-dysferlin participates in protein vesicle trafficking (FIG. 9).

Experimental data show that a “midi-dysferlin” protein, which comprises exon 40a, can be cleaved by calpain and is able to repair muscle membrane lesion, to restore myoblasts membrane protection from mechanical stress induced by osmotic shock and to participate in protein vesicle trafficking. Said “midi-dysferlin” truncated protein is necessary and sufficient to trigger membrane repair pathway after injury.

### Example 3: In Vivo Transfer of Midi-Dysferlin into a Murine Model for Dysferlinopathy

### Material and Methods

AAV9 vector containing the nucleic acid sequence according to the invention were injected intramuscularly in the left quadriceps of mice at 2 months of age. For this purpose, the animals were positioned in lateral decubitus position to clear access to the posterior loge of the quadriceps. Injections were performed with 25G*5/8 needles: injection of 20 μL of 1×PBS for control mice and injection of 20 μL of vectors diluted in 1×PBS to reach a concentration of 5.10¹¹ vg/mouse.

At 3 months mice injected and control were tested for their strength. The muscle strength of hind limbs of the mice was measured with the BIO-GS3 grip test (Bioseb). Mice were placed with hind limbs on the metal grid of the apparatus and slowly pulled backwards using their tails. The maximum tension was recorded, and the experiment was repeated 10 times for each mouse.

Following this test, mice were sacrificed, and muscle biopsy analyzed. Muscle sections were taken in a cryostat (ref CM1520, Leica, 7 μm) and fixed on superFrost Plus slides (ref 631-0108, VWR) through incubation with 4% paraformaldehyde for 10 minutes.

Hematoxylin and eosin (H&E) staining was performed by incubating for 2 minutes in hematoxylin (ref HHS32, Sigma-Aldrich), followed by washes with distilled water, and incubating for 10 minutes in eosin (ref HTAA0232, Sigma-Aldrich). The muscle sections were then incubated in 70% ethanol (once, 1 minute), 95% ethanol (once, 1 minute), 100% ethanol (twice, 1 minute), and in xylene (twice, 1 minute). Slides were stored at 4° C. until analysis under a microscope (Olympus BX51, 10× objective, Microvision). The percentage of centrally nucleated nuclei are monitored for each condition.

For detecting the presence of the midi-dysferlin we rely on immunofluorescence. The same muscle sections as above were permeabilized with a solution of 1×PBS and 0.5% Triton X-100 for 10 minutes. Blocking was performed for 1 hour at room temperature with the following solution: PBS 1×, BSA 5%, Donkey serum 5% and Triton X-100 0.1%. Then the dysferlin specific antibody was incubated according for one hour at 1/100 in the same buffer. Two washes with PBS 1× were performed, then the fluorescent antibody was incubated according to the manufacturer's recommendations.

Finally, two washes with 1×PBS were performed and the slides were mounted with Vectashield-Dapi (25 ng/mL, ref P36935, Life Technologies). The slides were stored at 4° C. until microscopic analysis (Zeiss Axio Imager Z2 microscope, 20× objective, ZEISS).

### Results

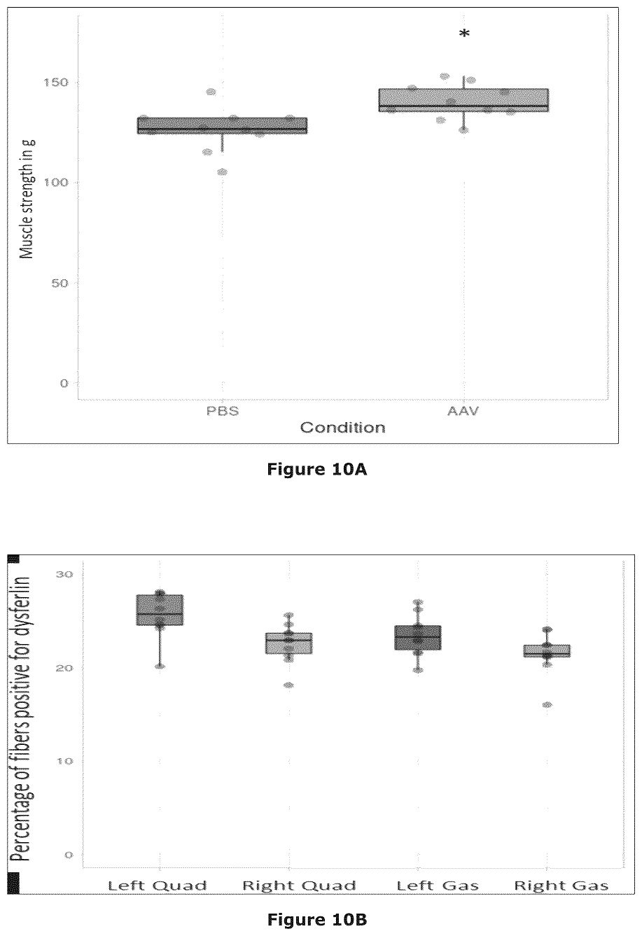

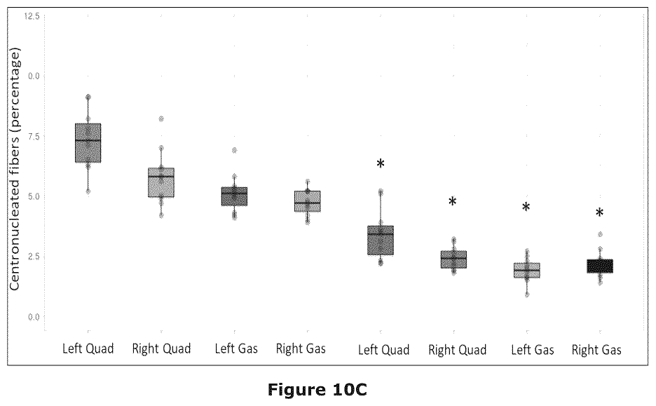

Experimental data clearly show that the administration in the Dysf mouse model of the midi-dysferlin construction according to the invention allows for: A significant increase in the strength developed by the AAV injected mice is detected compared to PBS-injected control mice (pvalue=0.0059) (FIG. 10A)

Immunofluorescence detection of dysferlin confirmed the presence of the midi-dysferlin protein in the muscle fibers of the injected mice. The amount of dysferlin detected is presented in FIG. 10B.

Histological analysis confirmed that midi-dysferlin presence in the injected muscles lowered the percentage of centronucleated cells in Dysf-treated fibers, compared to the PBS-injected control (FIG. 10C).

1-11. (canceled)

12. An isolated nucleic acid sequence comprising, or consisting of, from its 5′ to 3′ extremity:
a) a nucleic acid sequence encoding for the dysferlin amino acids No 1 to 301 (SEQ ID No 1), for an amino acid sequence exhibiting at least 80% identity with SEQ ID No 1, or for an amino acid sequence comprising at least 100 amino acids of SEQ ID No 1,
b) a nucleic acid sequence encoding for a polypeptide comprising a cleavage site by calpain, and
c) a nucleic acid sequence encoding for the dysferlin C2F, C2G and TM domains (SEQ ID No 2), for an amino acid sequence exhibiting at least 80% identity with said SEQ ID No 2 or for an amino acid sequence comprising at least 100 amino acids of SEQ ID No 2.

13. The isolated nucleic acid sequence according to claim 12, wherein said nucleic acid sequence encoding for at least 100 amino acids of the dysferlin N-terminal amino acids No 1 to 301 encodes for the amino acid sequence of C2A domain (SEQ ID No 3), or for an amino acid sequence exhibiting at least 80% identity with SEQ ID No 3.

14. The isolated nucleic acid sequence according to claim 12, wherein said nucleic acid sequence encodes for a polypeptide comprising a cleavage site by calpain, said polypeptide comprising or consisting of the amino acid sequence SEQ ID No 4, a functional fragment thereof comprising at least a sequence SEQ ID No 5, or an amino acid sequence exhibiting at least 80% identity with SEQ ID No 6.

15. The isolated nucleic acid sequence according to claim 12, wherein said nucleic acid sequence encoding for a cleavage site by calpain is nucleic acid sequence SEQ ID No 20, corresponding to dysferlin exon 40a, a nucleic acid sequence exhibiting at least 80% identity with SEQ ID No 20 or a nucleic acid sequence comprising at least 30 nucleotides of SEQ ID No 20.

16. The isolated nucleic acid sequence according to claim 12, comprising, from its 5′ extremity to its 3′ extremity:
i) a nucleic acid sequence encoding for the dysferlin C2A domain (SEQ ID No 3),
ii) a nucleic acid sequence corresponding to exon 40a (SEQ ID No 20), and
iii) a nucleic acid sequence encoding for the dysferlin C2F, C2G and TM domains (SEQ ID No 2).

17. The isolated nucleic acid sequence according to claim 12, which is SEQ ID No 21, or a nucleic acid sequence exhibiting at least 80% identity with SEQ ID No 21.

18. A recombinant vector comprising an isolated nucleic acid sequence according to claim 12.

19. A recombinant vector comprising an isolated nucleic acid sequence according to claim 12, which is a recombinant viral vector.

20. A pharmaceutical composition comprising a recombinant vector according to claim 18, and a therapeutically acceptable carrier, diluent or excipient.

21. A method of treating dysferlinopathy, comprising administering to a subject in need thereof a therapeutically effective amount of the recombinant vector according to claim 18.

22. The method according to claim 21, wherein said dysferlinopathy is a myopathy chosen among: Miyoshi myopathy (MM) and limb girdle muscular dystrophy type 2B (LGMD2B or LGMDR2).
